(19) United States
(12) Patent Application Publication
(10) Pub. No.: US 2024/0075343 A1
(43) Pub. Date: Mar. 7, 2024

(54) SYSTEM TO DETERMINE THE PHYSICAL FITNESS LEVEL OF A SUBJECT AND PROVIDE A CORRESPONDING EXERCISE ROUTINE FOR SAID SUBJECT AND METHOD OF USE.

(71) Applicant: Damien Young, Richboro, PA (US)

(72) Inventor: Damien Young, Richboro, PA (US)

(21) Appl. No.: 17/947,523

(22) Filed: Nov. 2, 2022

Related U.S. Application Data
(60) Provisional application No. 63/374,623, filed on Sep. 6, 2022.

(51) Int. Cl. A63B 24/00; A61B 5/00; A63B 71/06

(52) U.S. Cl. CPC A63B 24/0062; A61B 5/4872; A63B 24/0006; A63B 24/0075; A63B 71/0622; A63B 2024/0065; A63B 2071/0636; A63B 2230/75

(57) ABSTRACT

A system which determines a physical fitness level and an exercise and nutrition routine for the subject. The system comprises a non-transitory computer-readable medium encoded with computer executable instructions coupled to a processor, image system and display. The image system captures a front and side image of the subject's body. The system comprises a human machine interface whereby the subject's biographic data is entered into the system and is stored and retrieved from the computer medium. The system determines measurements of the subject's body, such as circumference measurements of the torso, neck, and a limbs from the images. The subject's physical fitness level determination is based on the biographic data and the body measurement developed from the images. An exercise routine and nutrition records database comprising a plurality of exercise and nutrition routine records filters said records based on filter criteria comprising the subject's biographic data and physical fitness level to provide at least one exercise and nutrition routine for the subject which is displayed for the subject.

### CROSS-REFERENCE TO RELATED APPLICATION

The present application claims priority from U.S. Provisional Patent Application Ser. No. 63/374,623, which is incorporated herein by reference in its entirety.

### BACKGROUND OF THE INVENTION

### Field of the Invention

This summary is an introduction of concepts which are further discussed in the Detailed Description. This summary is not intended to identify key features or essential features of the claimed subject matter, nor is it intended to be used to limit the scope of the claimed subject matter.

### Background

In one embodiment the current invention relates to a system that determines the physical fitness level of a subject and prescribes a corresponding exercise and nutrition routine for said subject.

### SUMMARY OF THE INVENTION

Other aspects, features, and advantages of the present invention will become more fully apparent from the following detailed description, the appended claims, and the accompanying drawings in which like reference numerals identify similar or identical elements.

### BRIEF DESCRIPTION OF THE DRAWINGS

FIG. 1 shows a side image of a subject's body according to a first exemplary embodiment of the present invention.

FIG. 2 shows a frontal image of a subject's body according to a first exemplary embodiment of the present invention.

FIG. 3 shows a process flow diagram of at least one of a physical fitness level determining and exercise routine and nutrition routine development system according to a first exemplary embodiment of the present invention.

FIG. 4A shows user or subject 200's extracted side contour image with clothing contours included according to a first exemplary embodiment of the present invention.

FIG. 4B shows user 200's side contour edge image without clothing contours included according to a first exemplary embodiment of the present invention.

FIG. 4C illustrates user 200's extracted front contour image with clothing contours included according to a first exemplary embodiment of the present invention.

FIG. 4D illustrates user 200's front contour edge image without clothing contours included according to a first exemplary embodiment of the present invention.

FIG. 5 illustrates line segments drawn between pose landmark points applied to an exemplary frontal image according to a first exemplary embodiment of the present invention.

FIG. 6 illustrates line segments drawn between various pose landmark points applied to the subject's side image according to a first exemplary embodiment of the present invention.

FIG. 7 illustrates line segments drawn between various pose landmark points applied to the subject frontal image according to a first exemplary embodiment of the present invention.

FIG. 8 shows a boundary applied to the subject's side image as well as body part measurement locations applied to the subject's side image according to a first exemplary embodiment of the present invention.

FIG. 9 illustrates a boundary applied to the subject's frontal image as well as body part measurement locations applied to the subject's frontal image according to a first exemplary embodiment of the present invention.

FIG. 10 illustrates 10 exemplary measurement locations on a subject's body applied to the subject's frontal image according to a first exemplary embodiment of the present invention.

FIG. 11 illustrates 10 exemplary measurement locations on a subject's body illustrated applied to the subject's side image according to a first exemplary embodiment of the present invention.

FIG. 12 illustrates an exemplary 10 predicted circumference measurements of a subject's body part measurements according to a first exemplary embodiment of the present invention.

FIG. 13 illustrates the first page of the subject's biographic data entry interface as a health questionnaire according to a first exemplary embodiment of the present invention.

FIG. 14 illustrates the second page of the subject's biographic data entry interface as a health questionnaire according to a first exemplary embodiment of the present invention.

FIG. 15 illustrates the third page of the subject's biographic data entry interface as a health questionnaire according to a first exemplary embodiment of the present invention.

FIG. 16 illustrates the fourth page of the subject's biographic data entry interface as a health questionnaire according to a first exemplary embodiment of the present invention.

FIG. 17 illustrates the last page of the subject's biographic data entry interface as a health questionnaire according to a first exemplary embodiment of the present invention.

FIG. 19 illustrates at least one portion of a first body fitness determining system, such as a body fat percentage, with at least one of the subject's height and weight and gender and a subject's side and a subject's frontal images as the system inputs according to a first exemplary embodiment of the present invention.

FIG. 20 illustrates the first page of the subject's body part measurements and calculations according to a body fitness determining system with the subject's height and weight and gender and side and frontal images as the system inputs system according to a first exemplary embodiment of the present invention.

FIG. 21 illustrates the second page of the subject's body part measurements and calculations according to a body fitness determining system with the subject's height and weight and gender and side and frontal images as the system inputs system according to a first exemplary embodiment of the present invention.

FIG. 22 illustrates the third page of the subject's body part measurements and calculations according to a body fitness determining system with the subject's height and weight and gender and side and frontal images as the system inputs system according to a first exemplary embodiment of the present invention.

FIG. 23 illustrates the fourth page of the subject's body part measurements and calculations according to a body fitness determining system with the subject's height and weight and gender and side and frontal images as the system inputs system according to a first exemplary embodiment of the present invention.

FIG. 24 illustrates the fifth page of the subject's body part measurements and calculations according to a body fitness determining system with the subject's height and weight and gender and side and frontal images as the system inputs system according to a first exemplary embodiment of the present invention.

FIG. 25 illustrates the last page of the subject's body part measurements and calculations according to a body fitness determining system with the subject's height and weight and gender and side and frontal images as the system inputs system according to a first exemplary embodiment of the present invention.

FIG. 26 illustrates the first page of a body fitness level determining system, such as a score, according to a first exemplary embodiment of the present invention.

FIG. 27 illustrates the second page of a second body fitness level determining system, such as a score, according to a first exemplary embodiment of the present invention.

FIG. 28 illustrates the third page of a body fitness level determining system, such as a score, according to a first exemplary embodiment of the present invention.

FIG. 29 illustrates the fourth page of body fitness level determining system, such as a score, according to a first exemplary embodiment of the present invention.

FIG. 30 illustrates a first page of a body fitness workout level determining system, such as a score, according to a first exemplary embodiment of the present invention.

FIG. 31 illustrates a second page of a body fitness workout level determining system, such as a score, according to a first exemplary embodiment of the present invention.

FIG. 32 illustrates a first page of a body fitness wellness level determining system, such as a score, according to a first exemplary embodiment of the present invention.

FIG. 33 illustrates a second page of a body fitness wellness level determining system, such as a score, according to a first exemplary embodiment of the present invention.

FIG. 34 illustrates a third page of a body fitness wellness level determining system, such as a score, according to a first exemplary embodiment of the present invention.

FIG. 35 illustrates a workout routine database according to a first exemplary embodiment of the present invention.

FIG. 36 illustrates additional exercise information of a workout routine database according to a first exemplary embodiment of the present invention.

FIG. 37 illustrates nutritional calculations such as used to provide a nutritional recommendation to a subject according to a first exemplary embodiment of the present invention.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Also for purposes of this description, the terms “index,” “value,” “rating,” “score,” “basis,” “level,” “formula,” “formulae,” “guide,” “indicia,” “indicant,” “ratio,” “indicator,” “calculation,” “calculator” refer to any manner known in the art or later developed wherein an indicator, sign, or the measure of something is provisioned. Additionally, the interposition of one or more additional elements may be contemplated, although not required.

Additionally for purposes of this description the terms “connected,” “linked,” “associated,” “united,” “coupled,” “joined,” “combined,” “banded,” others not mentioned here, refer to any manner known in the art or later developed wherein any item or items are brought together into contact or association in some respect. Additionally, the interposition of one or more additional elements may be contemplated, although not required. Conversely, the terms “directly coupled,” “directly connected,” etc., imply the absence of such additional elements.

Additionally, for purposes of this description, the terms “process,” “step,” “steps,” “system,” “method,” “operation,” may be used interchangeably and are used to describe a method of performing at least one a series of actions and steps to generally or specifically achieve a particular result.

Additionally, for purposes of this description, the terms “neural,” “computer,” “computer system,” neural system,” “smart device,” others not mentioned here may be used interchangeably to describe any electronic system capable of processing, transmitting, receiving, storing information.

Additionally, “measurement,” “measurements,” “dimension,” “dimensions,” may be used interchangeably and may refer to the measuring of a distance by a measuring device such as at least one ruler, scale, visual system measuring device, caliper, skinfold calipers, ultrasonic measuring device, others not mentioned here.

Additionally, for purposes of this description, the terms “data,” “output” “datum,” “input,” “output,” “link,” others not mentioned here are used in regards to the transmission, transmitting, carrying, information such as at least one datum.

For additional purposes of this description, the term “comparator” is used to describe a processor device that provides at least of one of a “correlation,” “comparison,” “evaluation,” “analysis”, “statistical analysis,” as part of an operation or process.

For additional purposes of this description, the term “characterize” is used to describe an operation that provides at least of one of “calculate” “defining,” “redefining,” “quantifying,” “outlining,” “identifying,” “portraying,” “factorizing,” “indicating,” “describing,” others not mentioned here.

For additional purposes of this description, the term “calculator,” or “calculation,” is used to describe an operation that provides at least of one of “evaluating,” “comparing,” “analyzing,” “statistically analyzing,” “contrasting,” “appraising,” “indicating,” “inspecting,” others not mentioned here.

For additional purposes of this description, at least one of the term “string” or “data” may be used to describe at least of one of an “input,” “connection,” “data point,” “datum,” “link,” “series,” others not mentioned here.

For additional purposes of this description, the term “input” may be used to describe at least of one of an “input,” “subject's input,” input from a “subject interface,” or “user interface,” “UI”, “data point,” “datum,” others not mentioned here.

For additional purposes of this description, the term “body fat percentage,” may be used to describe at least one of a calculation, formula, method, system operation that provides at least of one of “defining,” “redefining,” “outlining,” “identifying,” “portraying,” “factorizing,” “indicating,” “describing,” others not mentioned here, a representation of the amount of fat percentage contained on at least one of a subject's body and a human body.

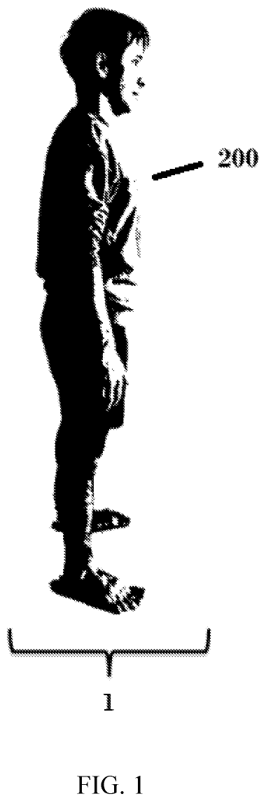

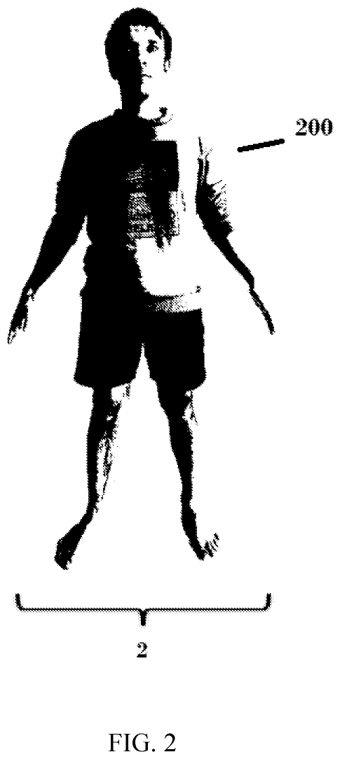

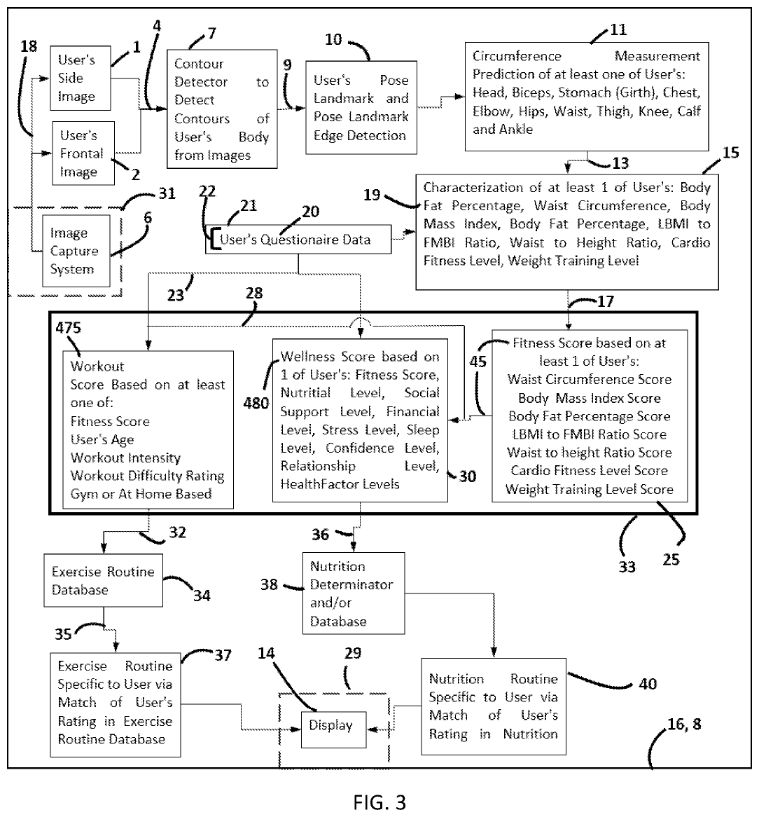

Referring FIGS. 1 and 2 respectively, a subject's side image 1 and a subject's frontal image 2 are shown for subject 200, respectively. The subject's side image 1 and the subject's frontal image 2 are one of developed by and provided to system and method 100 of FIG. 3 such as via image capture system 6 of FIG. 3. Image capture system 6 may be at least one of a camera and a smart phone camera and others not mentioned here. The term user and subject of system and method 100 are used interchangeably herein, such as user 200 and subject 200 of FIGS. 1 and 2. Frontal and/or frontal image, such as frontal image 2 of FIG. 2, at least one of shows and captures at least one portion of a front portion of a user's body, such as user 200 in this first exemplary embodiment. Side and/or side image, such as side image 1 of FIG. 1, at least one of shows and captures at least one portion of a side portion of a user's body, such as user 200 in this first exemplary embodiment. In this first exemplary embodiment, an exercise routine may be at least one of a usual series of exercises that subject 200 performs on at least one of a schedule and particular time and randomized schedule. A routine may be the practice of performing regular exercises in an order and/or a randomized fashion.

Referring FIG. 3, system and method 100 operates thereupon a non-transitory computer-readable medium 8 encoded as described herein, such as with computer executable instructions, which may be processed by processor 16. A non-transitory computer media may be a computer-readable media that can provide at least one of storing data for periods of time and operating in the presence of a memory device such as random access memory (RAM) and/or other powered storage devices. An example of being encoded may include being and/or having been converted into a form and/or particular form, others not mentioned here. Computer executable instructions may include computer instructions, such as included in a file and/or program, that can be performed and/or executed by a computer, such as an at least one of an *.exe and *.com and *.bat filetype, and others not mentioned here. An example of computer software executable code may be comprised of at least one of any object code and a machine code and any other code readable by a computer when loaded into memory and used directly by such computer to execute instructions. A processor, such as processor 16 may be computer logic circuitry which responds to and processes basic instructions that drive a computer.

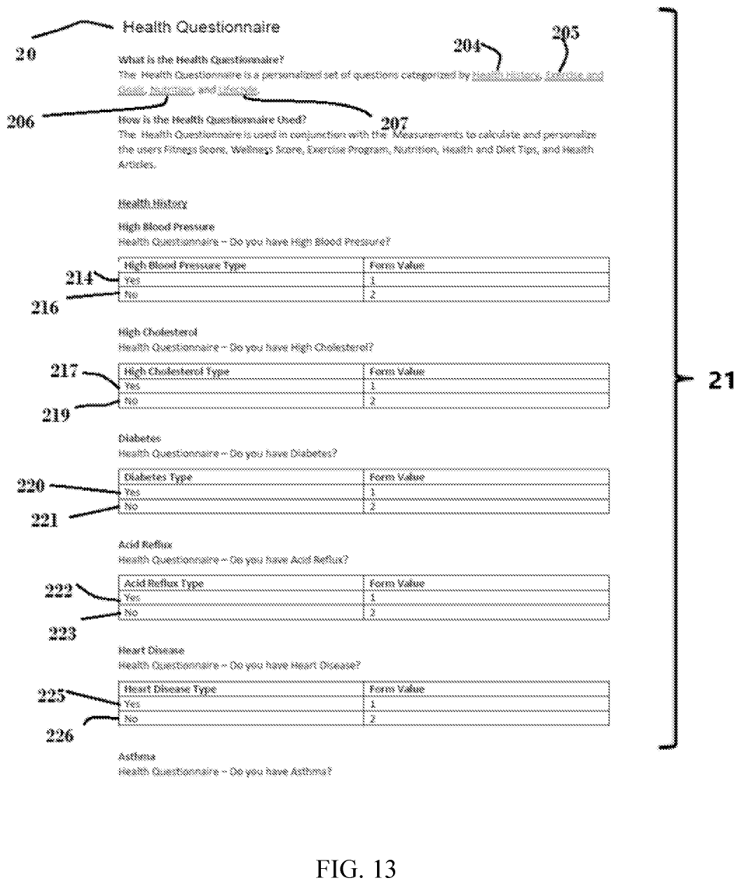

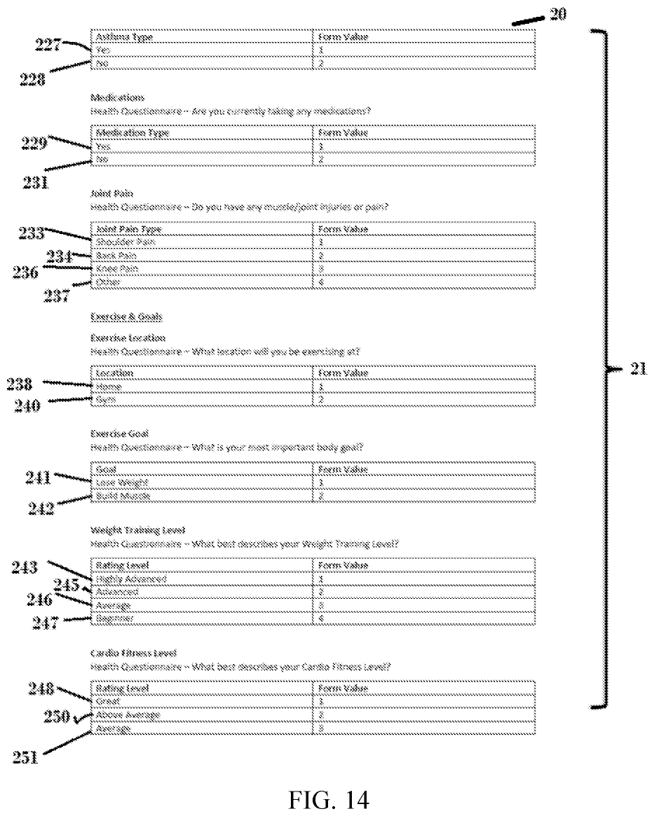

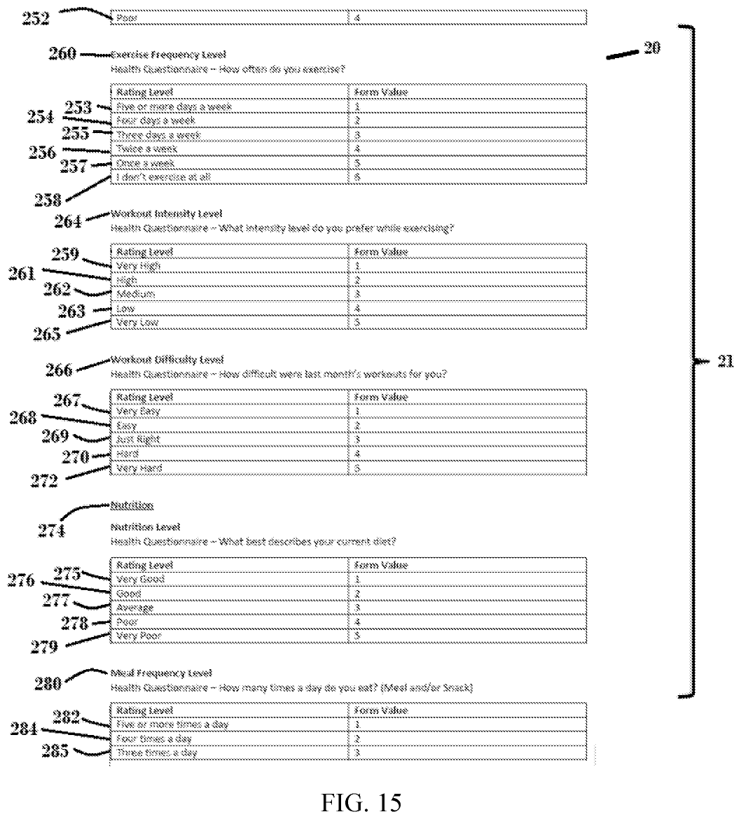

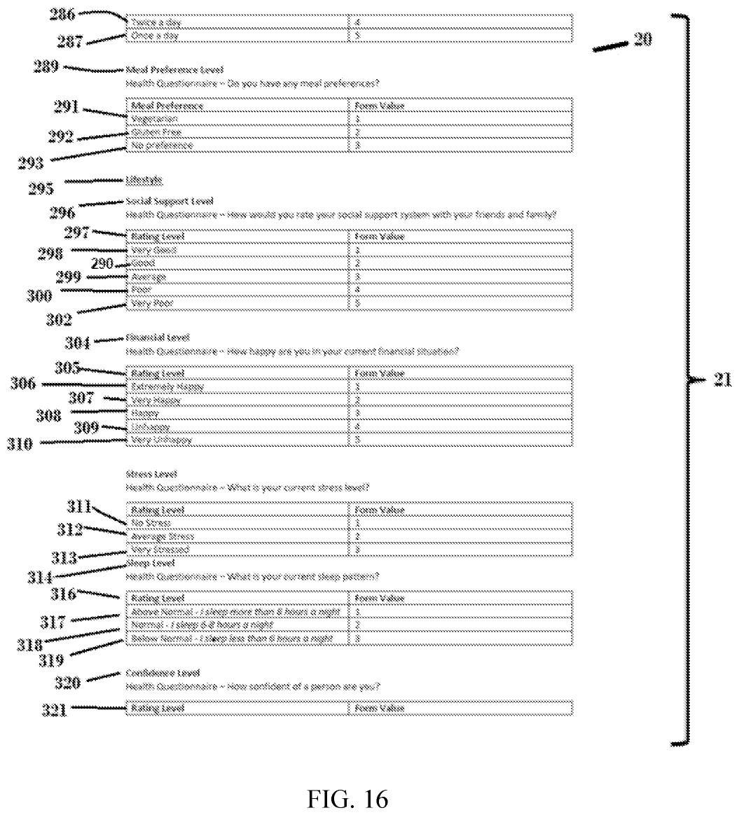

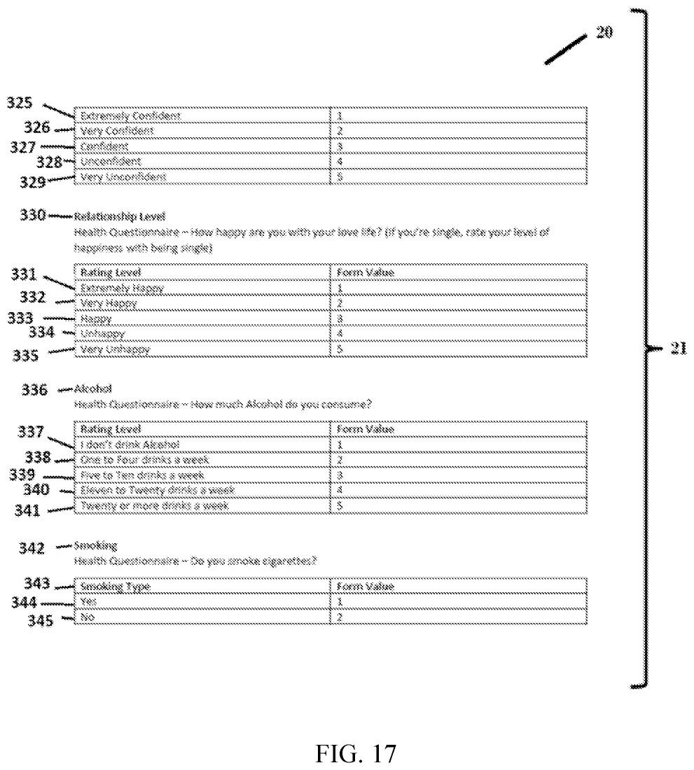

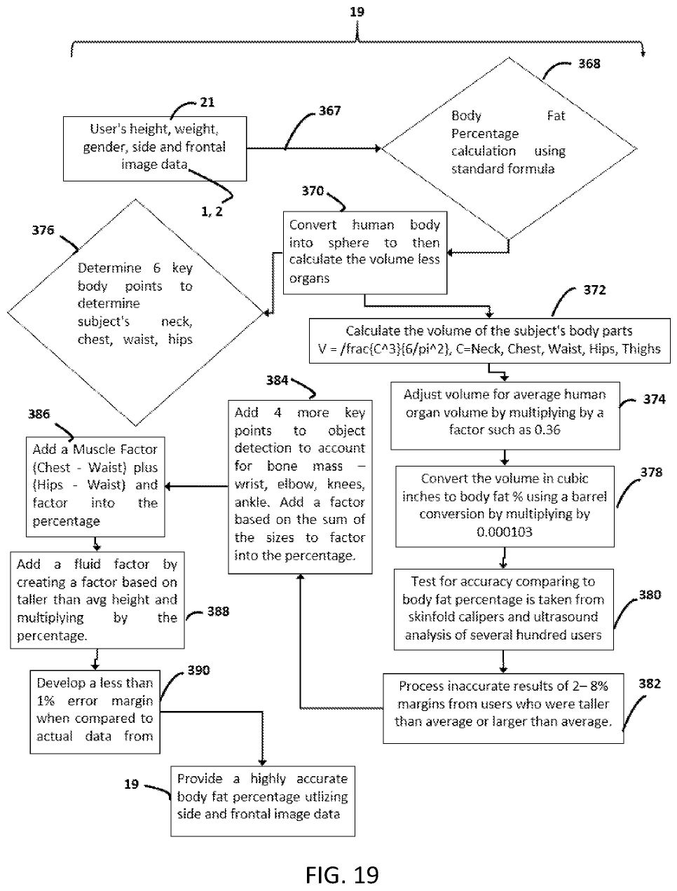

Referring FIGS. 1, 2, and 3, system and method 100 determines and/or provides the determining of subject 200's physical fitness level 32 and provides at least one of exercise routine 37 and nutrition routine 40 to user 200, such as via display 14. A subject or user of system and method 100, such as subject 200 of FIGS. 1 and 2, enters said subject's biographic data 21 in user's questionnaire data 20 through an interface, such as human machine interface (HMI) 22 of FIG. 3. Biographic data, such as subject 200's biographic data 21, may be at least one of stored and retrieved thereto and therefrom a non-transitory computer-readable medium, such as non-transitory computer-readable medium 8. Biographic data 21 may be comprised of at least one of information about an individual's history and behavioral patterns and demographics and physical characteristics and psychological characteristics and medical characteristics and others not mentioned here. A Human Machine Interface, or HMI, such as HMI 22 in the current exemplary embodiment, may be comprised of a user interface which connects a user, such as user 200 to at least one of a machine and system and device for information exchange relevant to system and method 100 and others not mentioned here. In the current exemplary embodiment, a physical fitness level for any user of system and method 100 may be at least one of a calculation and a prediction and an extrapolation and a measurement of any aspect of the user 200's body fitness. In this first exemplary embodiment, a measurement of user 200's body may provide at least one of the size and length and amount as established by measuring. Body fitness and/or user 200's physical fitness level may be comprised of at least one of a user's physical fitness and state of health and state of well-being and the ability to perform aspects of sports, occupations and daily activities and others not mentioned here. In the current exemplary embodiment, examples of types of biographic data, such as at least one of biographic data 21 and user's questionnaire data 20 is shown in FIGS. 13 through 18. In the current exemplary embodiment, the biographic inputs to method and system 100 of FIG. 3 to process the user's body fat percentage calculation 19 of FIGS. 3 and 19, such as provided by user's questionnaire data 21 of FIG. 3, are at least one of the subject's height, weight, gender and said subject's side image 1 and a subject's frontal image 2 for subject 200 of FIGS. 1 and 2. These inputs to system and method 100, the subjects height, weight, gender, side image and, front image, as shown in this first exemplary embodiment provide a highly accurate body fat percentage calculation 19, greatly reducing and/or eliminating the chance of erroneous input into system and method 100 of FIGS. 3 and 19.

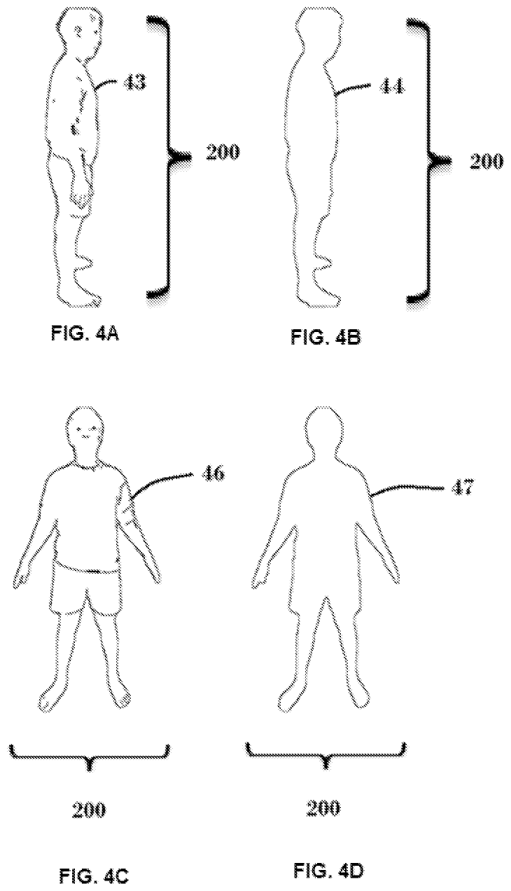

The process of system and method 100 comprises providing and capturing the subject 200's side and frontal images of FIGS. 1 and 2, such as by image capture 6 of FIG. 3. Image capture 6 may be one of integrally connected with system and method 100, and/or the function of image capture 6 may be provided by at least one of an image capture system connection to and the provision of image data to system and method 100, such as may be provided through possible system boundary 31 as shown in FIG. 3. Referring FIG. 3, captured image data 18 carries image data of user's side image 1 and user's frontal image 2 further carried by image data input 4 to image processor 7 whereby image contours are detected from said image data for the subject 200's body images, such as at least one portion of image data provided by user's side image 1 and user's frontal 2 of FIGS. 1, 2 and 3. The processing of image contour and image contour edge detecting, such as processed by system and method 100 are illustrated in FIGS. 4A-4D.

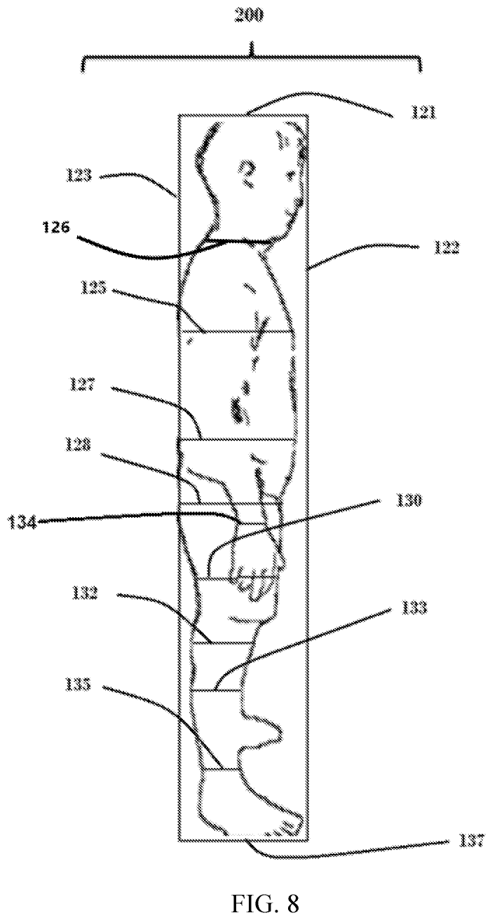

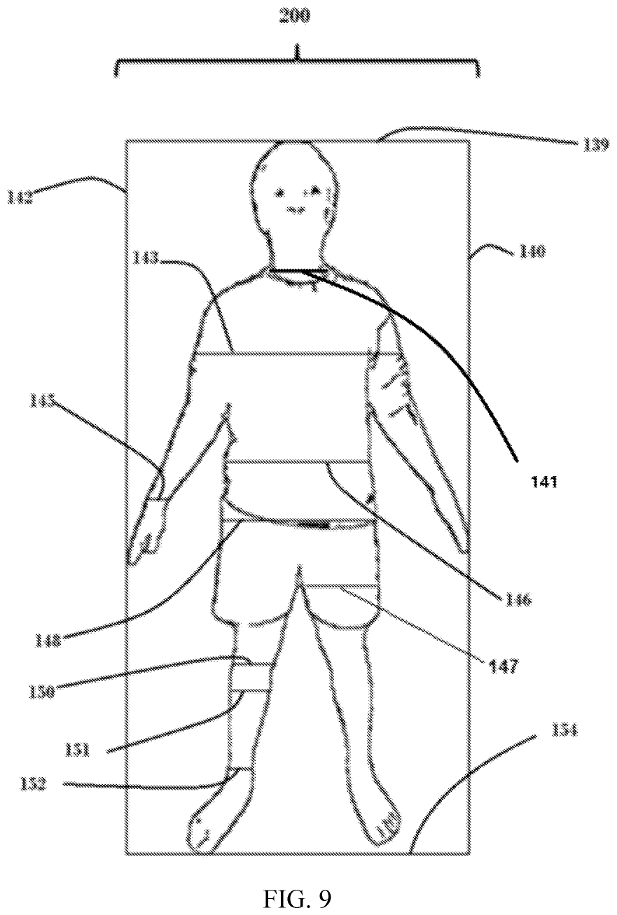

Referring FIG. 3, Data 9 carries image data to image pose landmarker 10 whereby user 200's pose landmark and pose landmark edges are detected and outputted to measurement predictor 11 FIG. 3. Measurement predictor 11 receives said image pose landmark and edge data and processes circumference measurement predictions 13 of at least one of the subjects and/or user 200's torso and abdomen and limbs and head and bicep and stomach and girth and chest and elbow and hip and waist and thigh and knee and calf and ankle as noted in FIG. 3 and herein. In this first exemplary embodiment, it is estimated that circumference measurement predictions 13 for subject 200's body may range from a minimum of 2 to 16 of said measurements, from said user's side image 1 and said user's frontal image 2, such as four measurements developed for subject 200's equivalent circumference waist and chest and thigh and hip measurements, whereby such measurements for subject 200 may correspond to measurement and/or circumference measurements taken from locations of user 200's body as illustrated by user's neck 126 and 141 and chest 125 and 143 and waist 127 and 146 and hip 128 and 148 and thigh 130 and 147 of FIGS. 8 and 9 respectively. As described herein, the term circumference may refer to at least one of circumference and equivalent circumference, whereby said at least one circumference and equivalent circumference may represent a measurement such as the boundary line of an area and/or object and the length of such a line, such as at least one of the linear distance of the perimeter and/or boundary of the referenced subject 200's body part shape and by approximating the linear distance of the perimeter of the referenced subject 200's body part shape and others not mentioned here, such as may be applied to at least one of subject 200's head and bicep and stomach and girth and chest and elbow and hip and waist and thigh and knee and calf and ankle and others not mentioned here. In addition, in this first exemplary embodiment, the term body part circumference measurement is used illustratively to show equivalent circumference measurement predictions for subject 200, and may also comprise any body part measurement of subject 200.

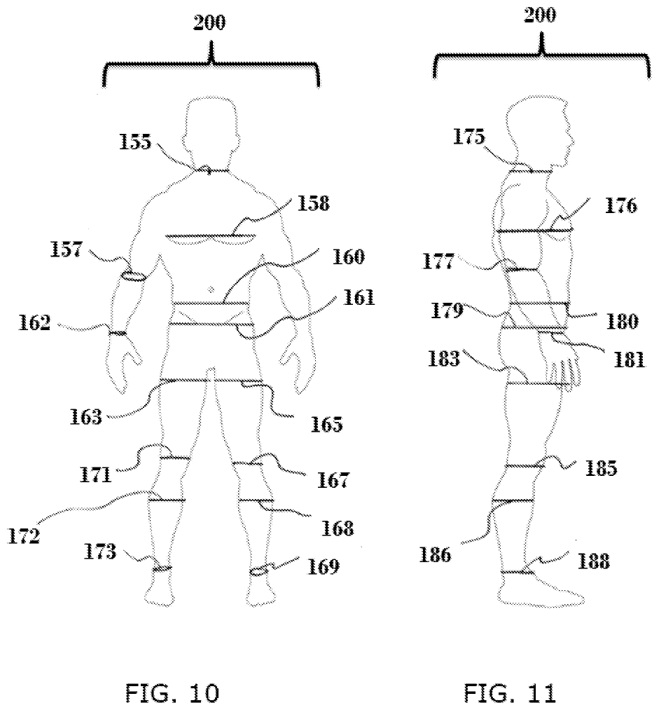

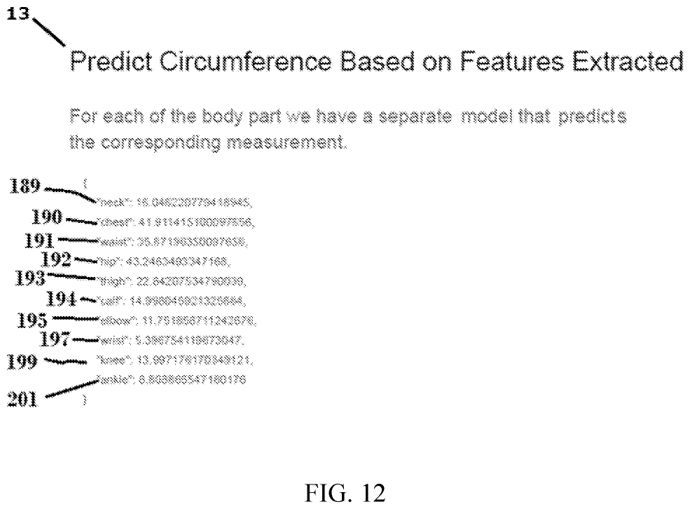

An additional illustration showing an example of 10 circumferential measurement prediction locations which may be developed by system and method 100 of FIG. 3, such as at least one of the circumference and the equivalent circumference derived from subject 200's neck 155 and 175 and chest 158 and 176 and waist 160 and 180 and hip 161 and 179 and thigh 163 or 165 and 183 and calf 172 and 168 and 185 and elbow 157 and 177 and wrist 162 and 181 and knee 171 and 167 and 185 and ankle 173 or 169 and 188 as shown in FIGS. 10 and 11 respectively. It is noted these measurements may be two dimensional or three dimensional.

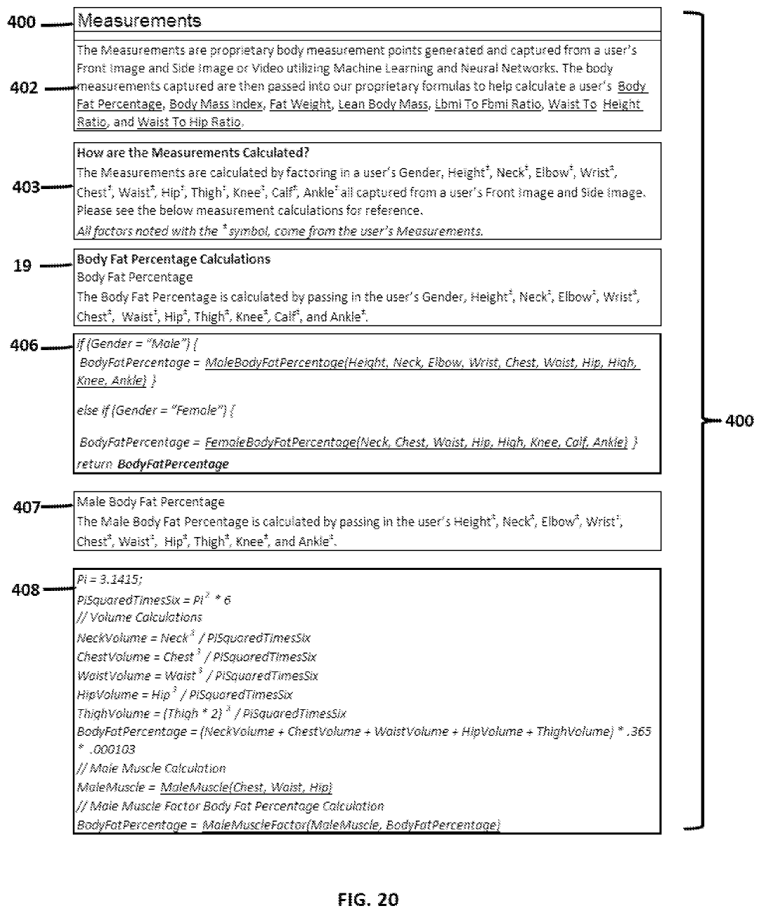

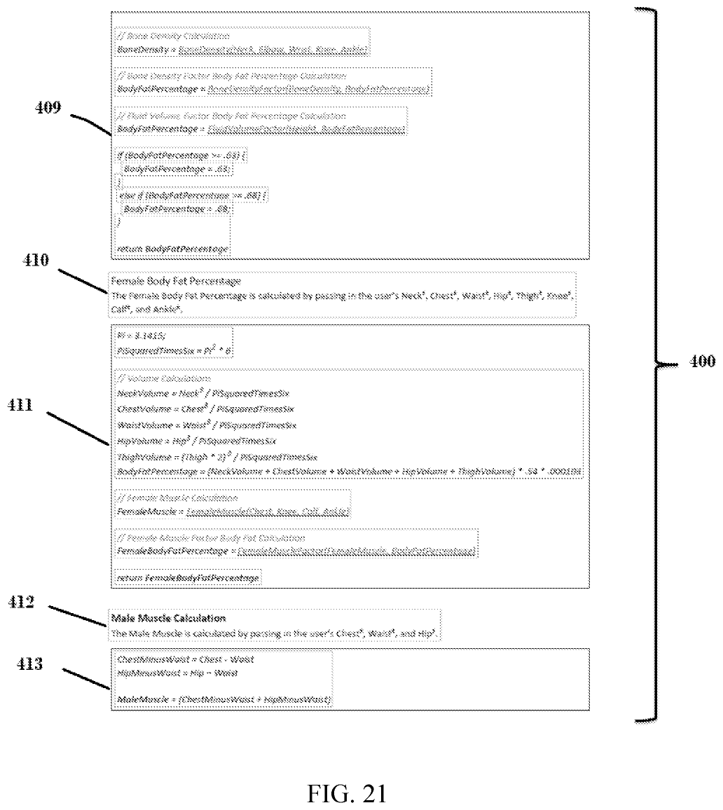

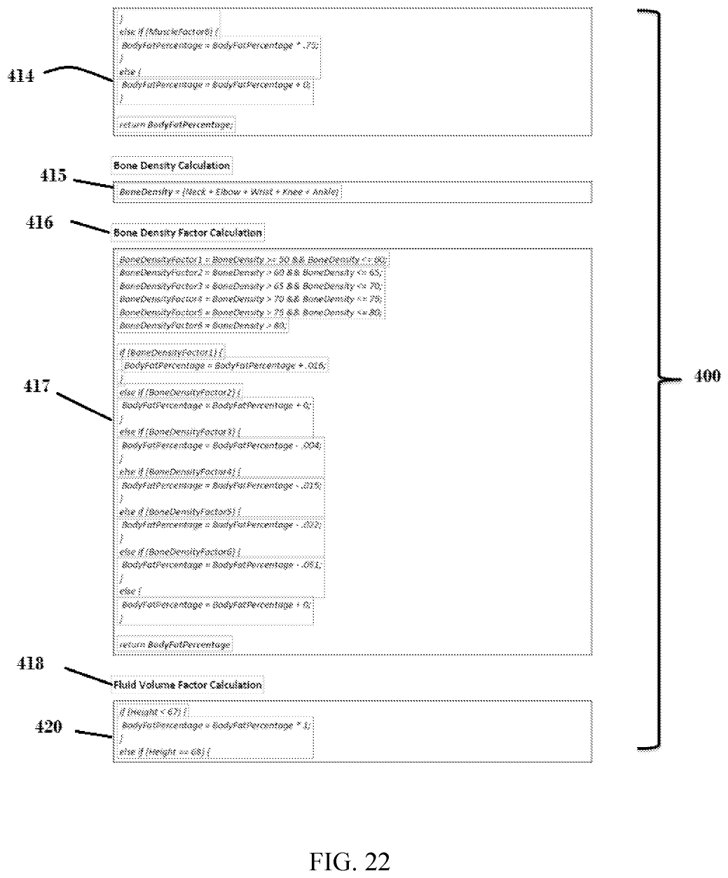

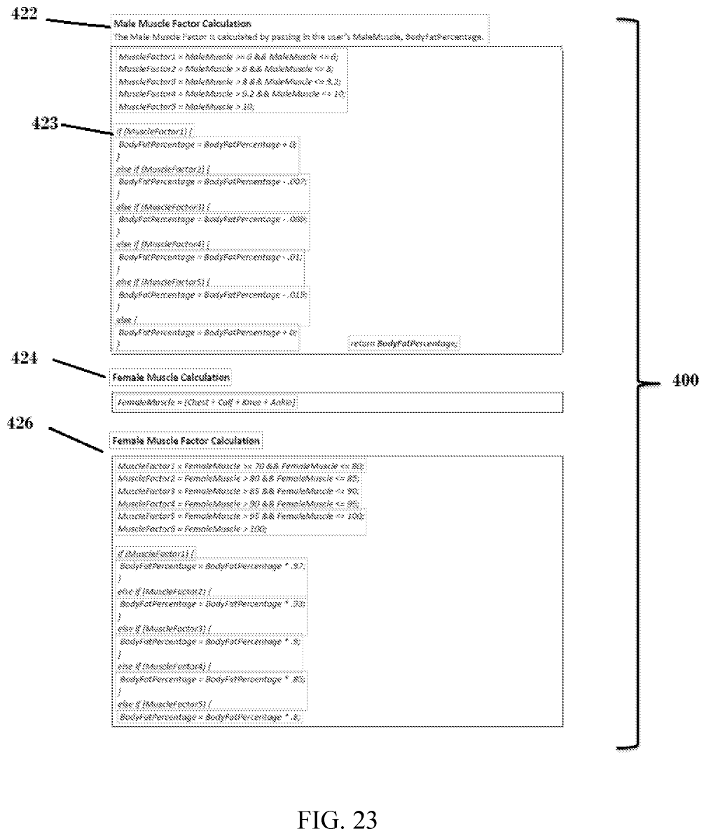

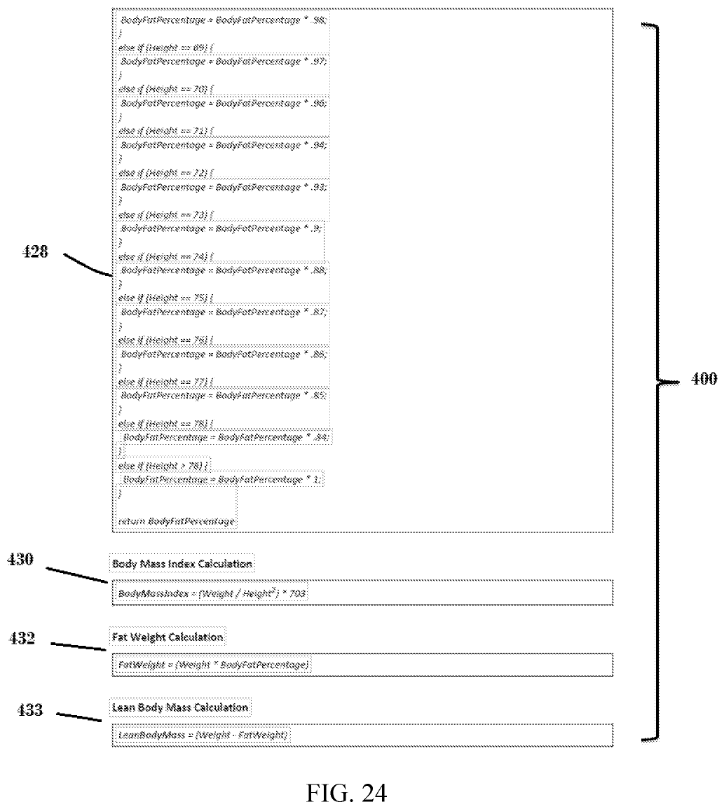

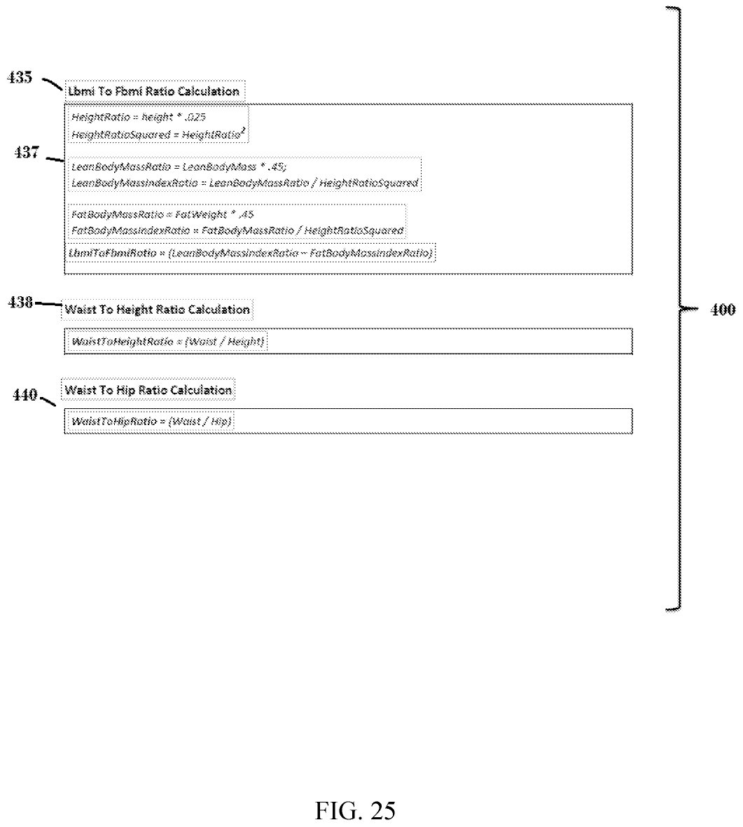

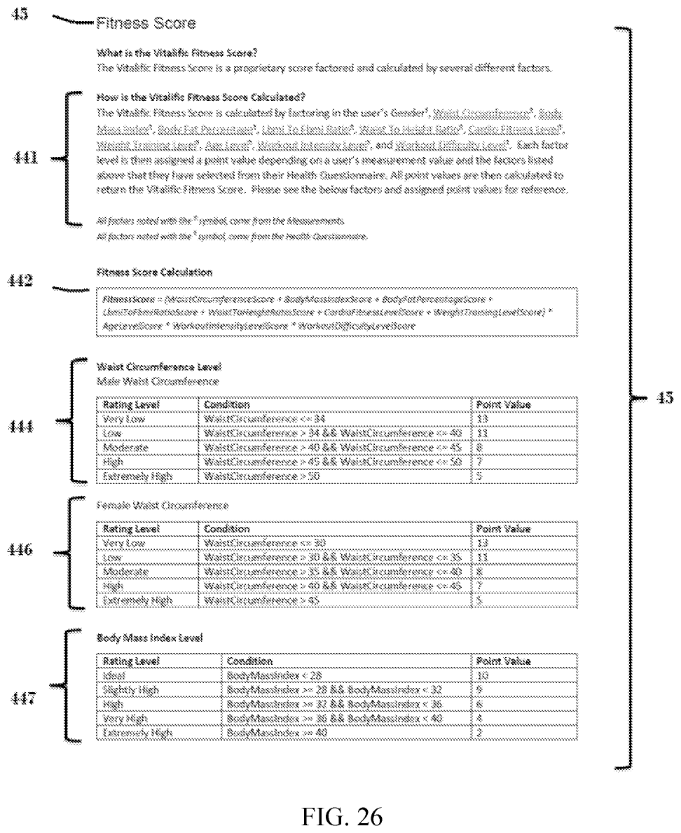

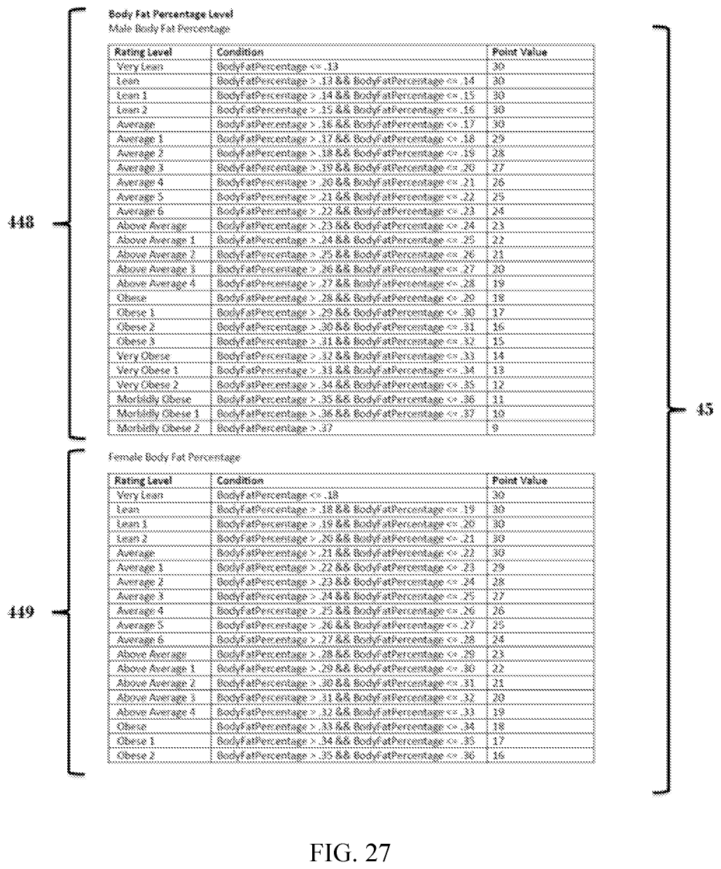

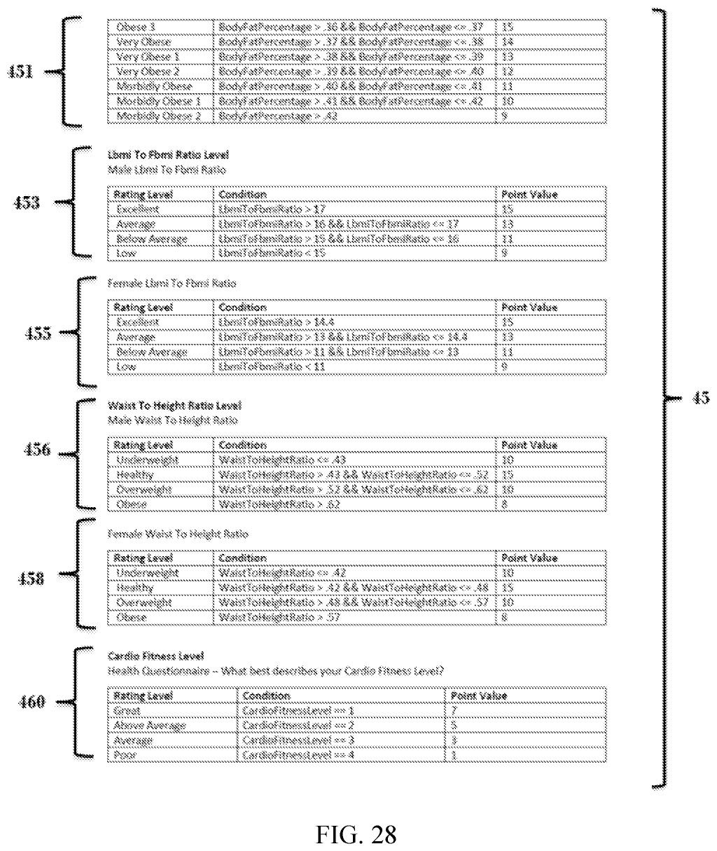

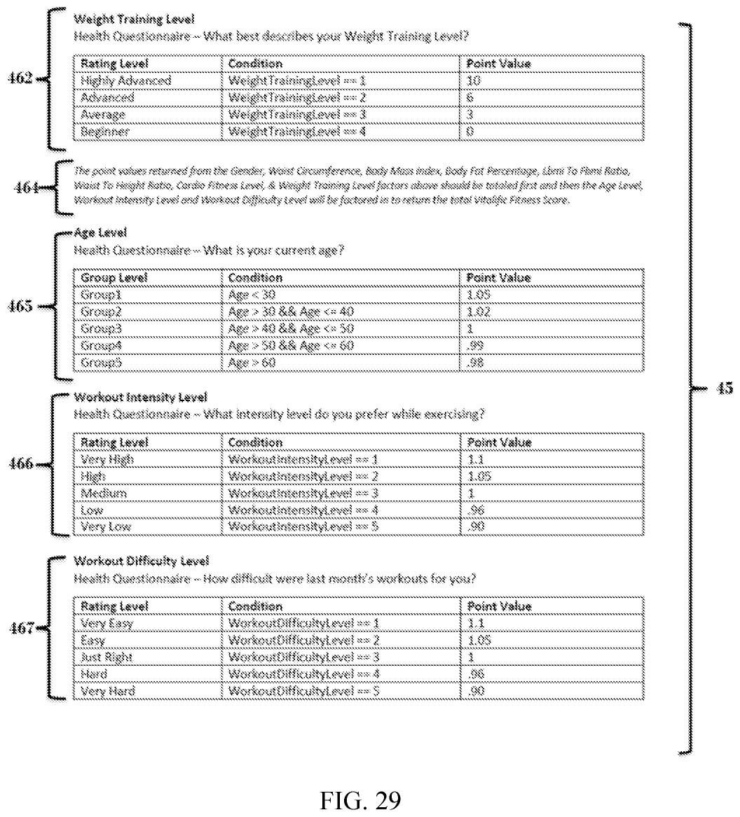

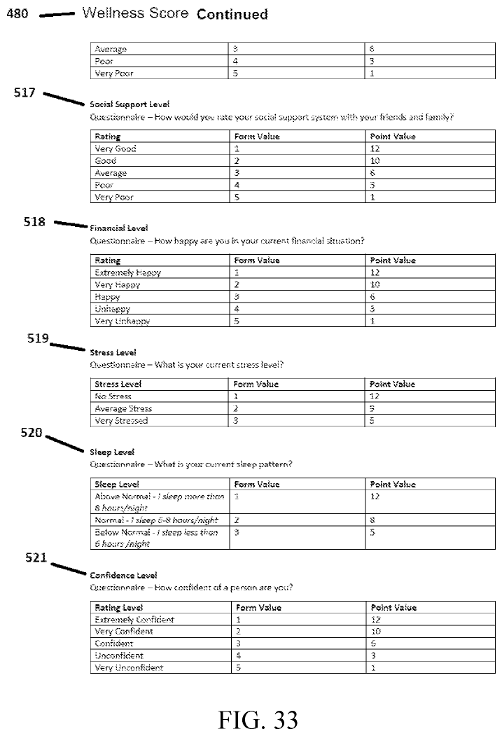

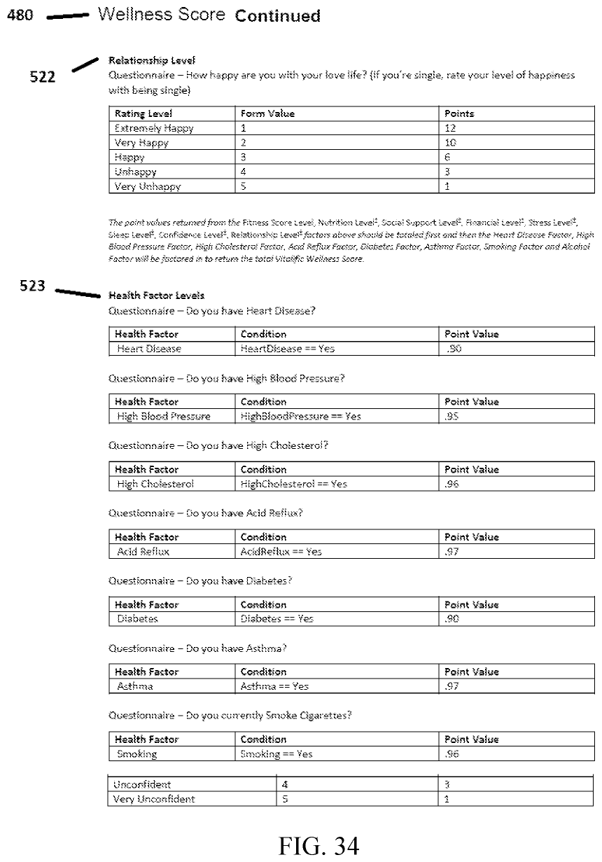

In the current exemplary embodiment, referring FIG. 3, the subject 200's predicted body part equivalent circumference measurements 11 are outputted as equivalent circumference measurements 13 and provided to fitness characterization 15. As shown in FIG. 3, characterizer 15 characterizes at least one of subject 200's physical fitness levels, such as body fat percentage calculation 19 of FIG. 19 and waist circumference 444 and 446 of FIG. 26 and body mass index 430 of FIG. 24 and Lean Body Mass Index (LBMI) 433 of FIG. 24 and waist to height ratio 438 of FIG. 25 and cardio fitness level 460 of FIG. 28 and weight training level 462 of FIG. 29 and age level 465 of FIG. 29 and others not mentioned here as described herein. Any determinations of user 200's fitness level by system and method 100, such as shown in FIGS. 3 and FIGS. 19-34, may provide at least one calculation and/or determination used to directly and/or indirectly influence user 200's body fitness determination as illustrated herein, such as by calculations and measurements 400 illustrated in FIGS. 20, 21, 22, 24, 25. Additionally, at least one data input for user 200, such as provided by at least one of biometric data 21 and user's questionnaire 20 of FIGS. 3, illustrated in FIGS. 13 through 18, may also be used to calculate and/or determinate any portion of any user 200's body fitness determination as illustrated herein and inferred herein. Additionally, any methodology and/or information such as illustrated by any portions of FIGS. 3, 13, 14, 15, 16, 17 and 19 may be utilized to fitness score determinator 25 to determine fitness score 45 as illustrated in in FIGS. 3, 26, 27, 28, 29. Fitness score and physical fitness level and fitness level and fitness rating may be used interchangeably herein. As defined herein, at least one of cardio fitness level and cardio fitness level score and cardio fitness score are used interchangeably. In this first exemplary embodiment, a cardio fitness level may be at least one of a qualitative and quantitative analysis of subject 200s cardio fitness, such as any determination of at least one of subject 200's cardiovascular fitness and aerobic fitness and referring to the ability of subject 200's body to take in and use oxygen while exercising, others not mentioned here.

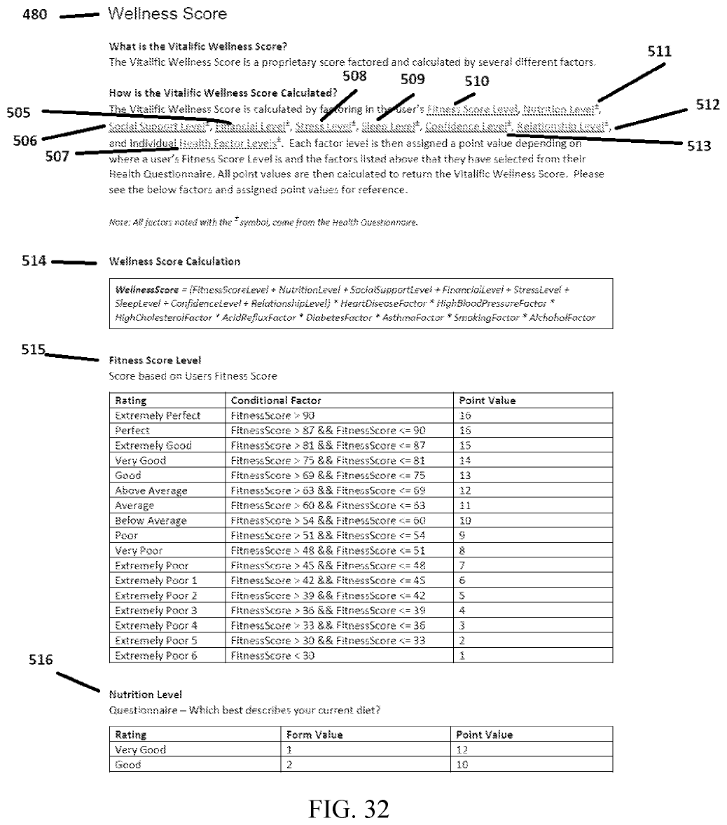

User's wellness fitness score 480 of FIGS. 3, 32, 33, 34 is determined through methodology such as illustrated by at least one of fitness score 45 of FIGS. 3, 26, 27, 28, 29, 32, 33 and 34, such comprising nutrition level 516 and social support level 517 and financial level 518 and stress level 519 and sleep level 520 and confidence level 521 and relationship level 522 and health factor level 523, others not mentioned here of FIGS. 32, 33, 34.

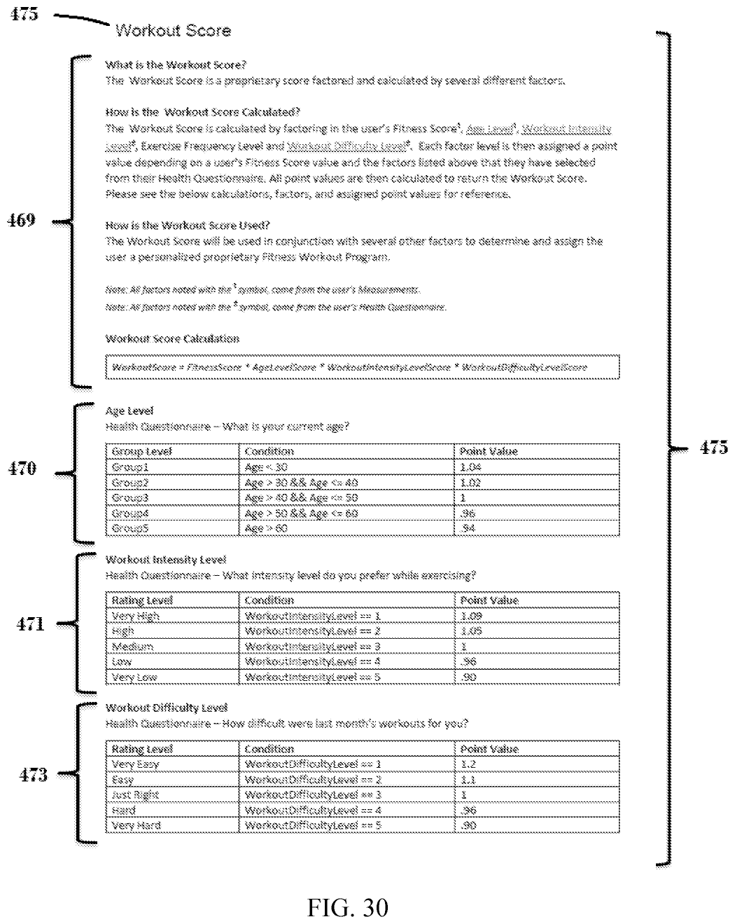

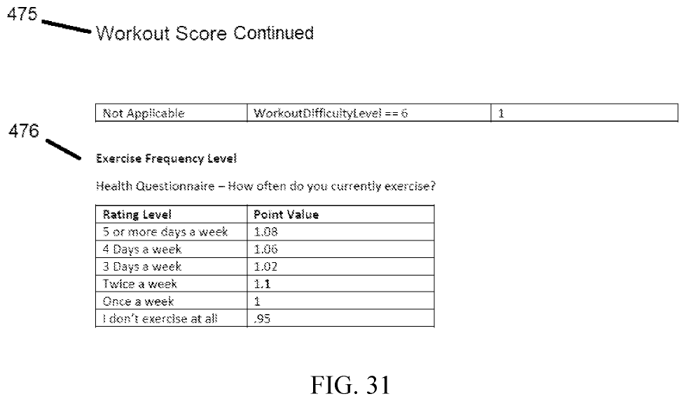

Referring FIG. 3, fitness score 45 is also provided to workout score determinator 475 via input 28, in this first exemplary embodiment, to develop user 200's fitness level 32 by determinator 475. In this first exemplary embodiment, fitness level 32 is illustrated as being based on at least one of fitness score 45 of FIG. 3 and user's age level 470 and workout intensity level 471 and workout difficulty rating 473 and exercise frequency rating 476 of FIGS. 30 and 31, and at least one portion of user 200's biographic data 21, such as whether or not a user's gym is home-based or not, others not mentioned here. Referring FIG. 3, process 33 is shown illustrating a combined fitness analysis methodology for subject 200, comprising the development of at least one of fitness score 45 and wellness score 480 and workout score 475 for user 200 of FIGS. 1 through 11. Process 33 illustrates fitness score 45 and wellness score 480 and workout score 475 as a combined process, however, fitness score determination 45 and wellness score 480 and workout score determination 475 may be performed at least one of singularly, simultaneously, separate, in order, out of order, integrally combined, others not mentioned here.

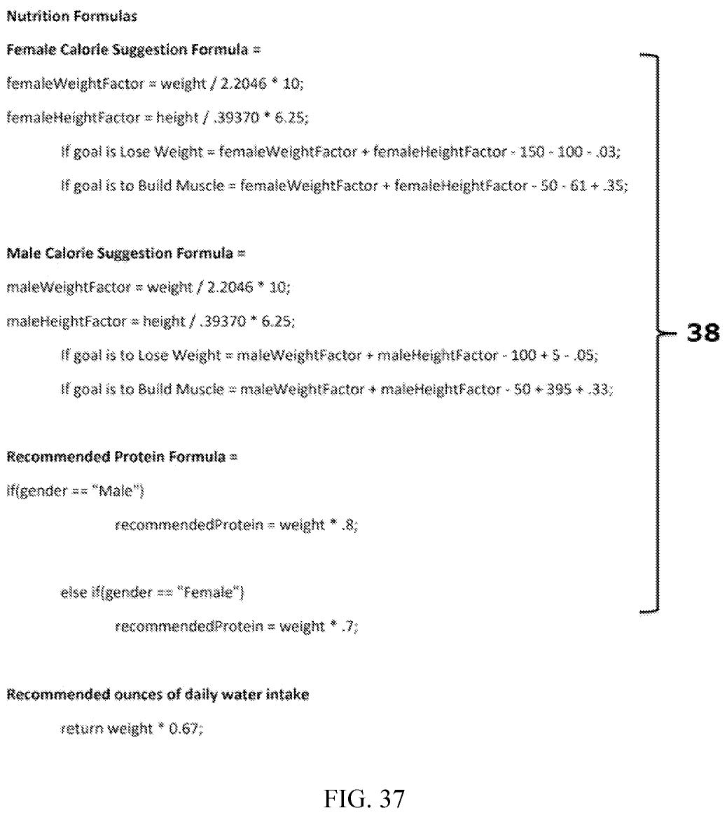

Referring FIG. 3, subject 200's physical fitness level rating 32 is provided to exercise routine records database 34 whereby database 34 will perform the assigning of an exercise routine for user 200 through data filtering. Data filtering of exercise routine records database 34, in this exemplary embodiment, refers to the process of filtering and/or data filtering the wide range of exercise routine records in database 34, said data filtering based on meeting certain filter criteria to output a refined range of exercise routine records, such as at least one exercise routine. As such, the exercise routine records database 34 exercise data routine records are refined by subject 200's specific information and/or criteria, excluding other exercise routine record information irrelevant to subject 200 as this process is performed. Nutrition determinator 38 in this first exemplary embodiment determined subject 200's nutrition routine through calculations such as illustrated in FIG. 37. Alternately, the use of data filtering of a nutrition routine records database could be performed by filtering a wide range of nutrition routine records in the database, and the data filtering could be based on meeting certain filter criteria to output a refined range of nutrition routine records, such as at least one nutrition routine for subject 200.

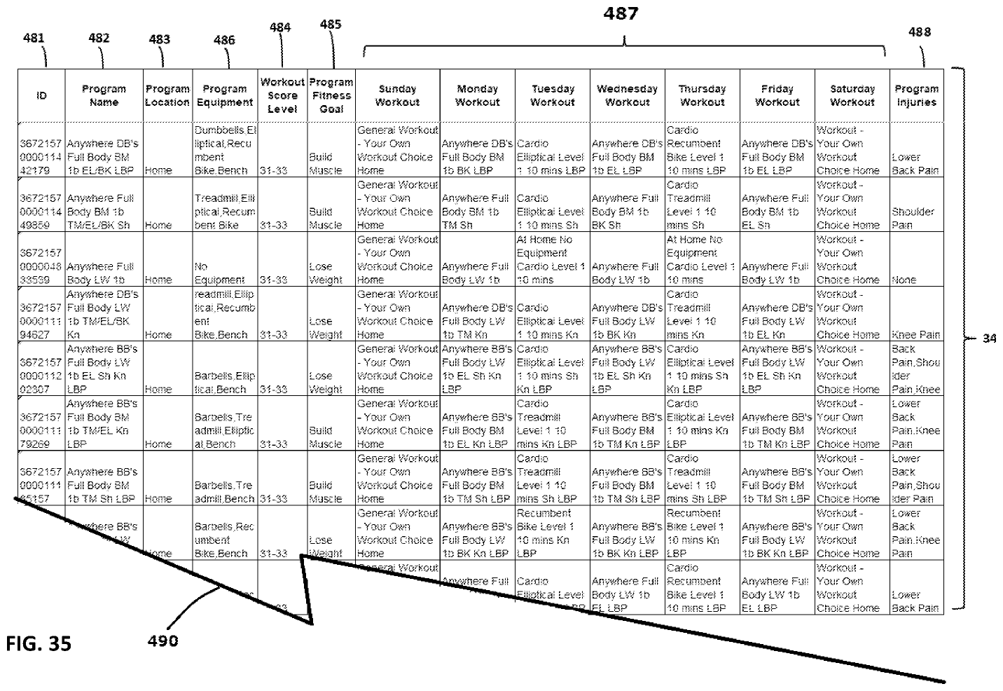

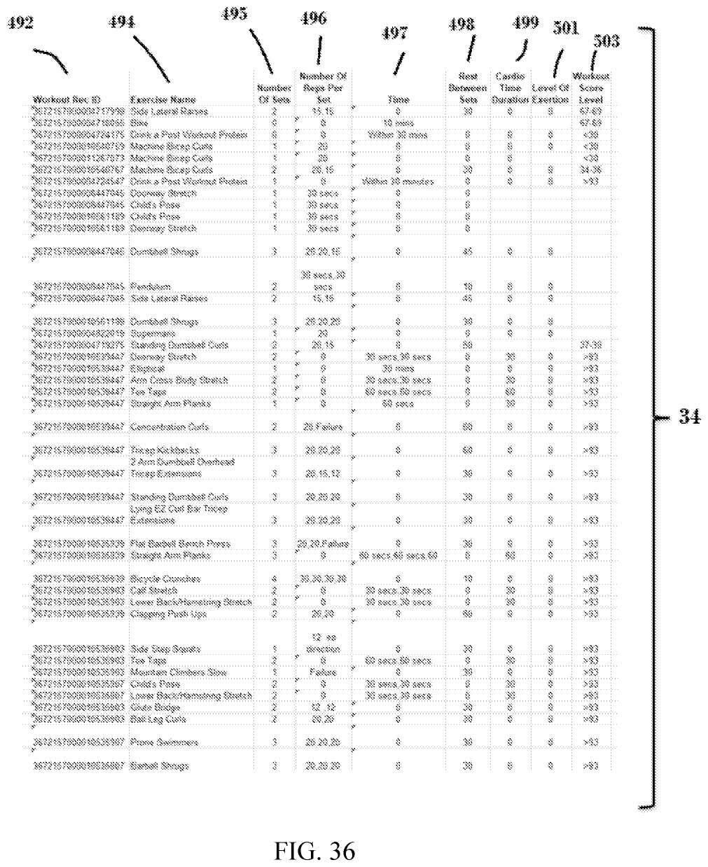

An illustration of an exemplary exercise routine database 34 is shown in FIG. 35 and FIG. 36. In addition, nutrition calculations, such as shown in FIG. 37, may represent nutrition calculations by system and method 100 of this first exemplary embodiment. As contained herein, physical fitness level and body fitness level and fitness score are used interchangeably. In addition, the term characterizing and determining may be used interchangeably.

Exercise routine database 34 of FIG. 3 illustrated in FIGS. 35 and 36 contains a plurality of exercise routine records, each exercise routine record in the current exemplary embodiment may comprise at least one of an identifier 481 and program name 482 and program location 483 and program equipment 486 and program workout score level 484 and program fitness goal 485 and exercise program schedule information 487 and program injuries 488 and others not mentioned here as shown in FIG. 35, whereby at least one of the data segments of exercise routine database 34 can filter data contained in database 34 to at least one exercise routine 37 for subject 200. An identifier, in this exemplary embodiment, provides identifiable information for identifying at least one of an exercise routine, and a nutrition routine record should a nutrition routine database be used instead of or in combination with nutrition determinator 38, such as a number, code, others not mentioned here. Additionally, as shown in FIG. 36, the current exemplary embodiment database 34 may also contain at least one of workout identifier 492 and exercise name 494 and number of sets 495 and number of reps per set 496 and time/duration 497 and rest between sets 498 and cardio time duration 499 and level of exertion 501 and workout score level 503. The at least one filtered exercise routine 37 for subject 200, such information illustrated in FIGS. 35 and 36, may then be displayed on display 14 of FIG. 3 in this first exemplary embodiment. An exemplary showing of a display, such as display 14, may be at least one of a computer readout and digital display and graphical display and liquid crystal display and an email display and text display and dynamic display and static display and any display of sound and any other communicative indicia not mentioned here.

Additionally, exercise database 34 of FIGS. 3 and 35, 36, and 37, and a nutrition database if used with system and method 100 could provide additional filtering of data of any exercise and/or nutritional routine corresponding to any portion of at least one of user 200's biographic data 21 and image data of image 1 and image 2 and information developed from image 1 and image 2 of FIGS. 1, 2, and 3, such as entered thereinto system and method 100. An exemplary showing of such biographic data may include user 200's fitness goal 485 and program equipment 486 and program location 483 and program injury 488 and others not mentioned here as illustrated in FIG. 35. In this first exemplary embodiment, equipment available to subject 200, such as exercise equipment available to subject 200, may comprise at least one of an exercise machine and weights and free weights and cardio equipment and exercise bikes and treadmills and any piece of equipment relevant to an exercise routine for subject 200. An injury status, such as for subject 200 as used by system and method 100 of FIG. 3, may be comprised of any injury data relevant to subject 200's body such as at least one of a back and spine and head and torso and leg and arm and shoulder and limb and hand and foot and ankle and knee and hip and other injures not mentioned here.

Referring FIG. 3 wellness score 480 is provided to nutrition determinator 38 via input 36. Referring FIG. 37 and FIG. 3. nutrition routine determinator 38 contains a plurality of calculations to determine subject 200's at least one nutritional diet, such as at considerations for least one of user 200's biographic data 21 and user 200's body fat percentage 19 and fitness score 45 and wellness score 480 to output at least one of a recommended calorie intake and protein intake and carbohydrate intake and fat intake and vitamin intake and others not mentioned here, for subject 200 of FIGS. 1 through 11.

Referring FIG. 3, at least one of nutrition routine 40 and exercise routine 37 is provided to display 14 for subject 200 of FIGS. 1 and 2. Display 14 may be at least one of internal and external to system and method 100 as indicated by possible boundary 29 of FIG. 3.

The measurement prediction processing of image data provided by subject's side image 1 and 2 of FIGS. 1-2 is illustrated through FIGS. 1-12. The measurement prediction process of system and method 100 begins with developing extracted images by instance segmenting of said images 1 and 2 into extracted side image and extracted frontal 4A and 4C, respectively. This extracted image data may be provided and/or processed through software, such as with at least one of open source software and PixelLib, whereby the extraction subject 200's body from images 1 and 2 is performed to provide extracted image 43 and 46 of FIGS. 4A and 4C. Further processing of extracted image 43 and 46 may comprise decoding of at least one of subject 200's side image 1 and frontal image 2 of FIGS. 1 and 2 and extracted images 43-46 of FIGS. 4B and 4D into red and green and blue matrices using software such as OpenCV may be performed to aid in the development of edge images 44 and 47 such as shown in 4B through 4D. In addition, edge detection, such as canny edge detection, may be performed on the extracted images 4A and 4C to provide subject 200's edge images such as subject 200's side and frontal posture images with inner and outer edges as shown in FIGS. 4B and 4D. Performing high quality monocular depth estimation via heat mapping of the images of 4A and 4C, as well as body instance segmentation with image depth computation may also be completed in the development of edge images 4B and 4D. As at least one of the high quality monocular depth estimation and the heat mapping and person instance segmentation with image depth computation may blur the posture of the desired edge images 44 and 47 outputs, whereby further performance of canny edge detection may be performed as part of the processing of the extracted images of FIGS. 4A and 4C into edge images 4B and 4D leaving only the clear edged outer contour and/or outer contours of subject's 200 images 44 and 47 of FIGS. 4B and 4D.

In figure FIG. 4A, subject 200's side extracted image 43 is shown with subject 200's clothing contours included. Referring FIG. 4B, user 200's side edge image 44 is shown without clothing contours. In figure FIG. 4C, user 200's front extracted image 46 is shown with user 200's clothing contours. Referring FIG. 4D, user 200's front edge image 47 is shown without clothing contours. In the current exemplary embodiment, edge images 44 and 47 of subject 200 of FIGS. 4A and 4D may be provided by at least one of system and method 100 and image processor 7 and image landmarker 10 of FIG. 3. As described herein, the term edge and contour may be used interchangeably. At least one portion of user 200's body contours as illustrated under in FIGS. 4A through 4D may be used as part of the development of measurements 13, such as body part circumference measurements, for subject 200's body.

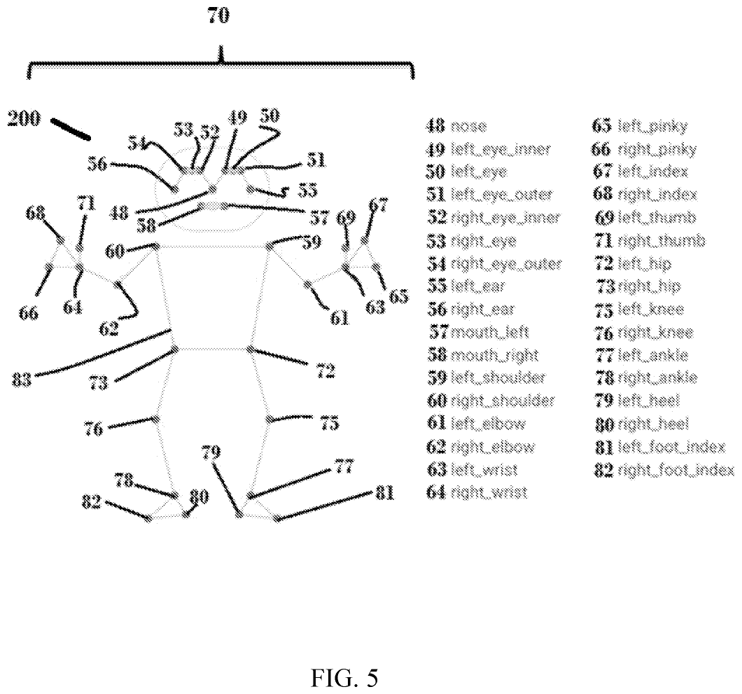

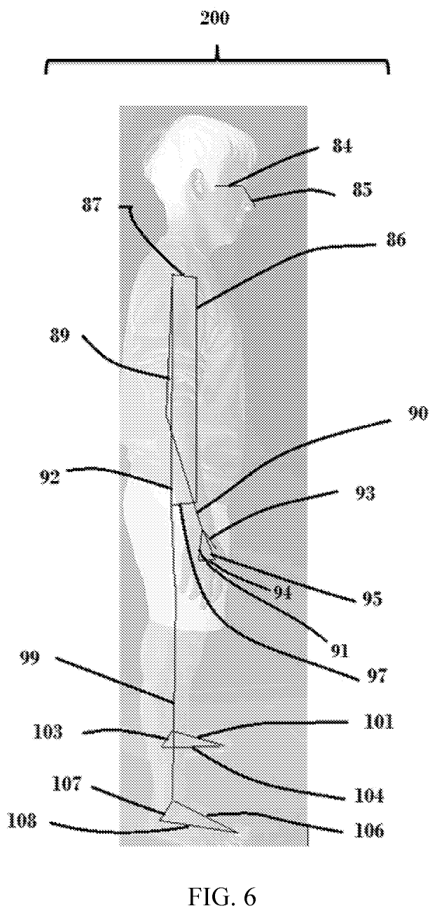

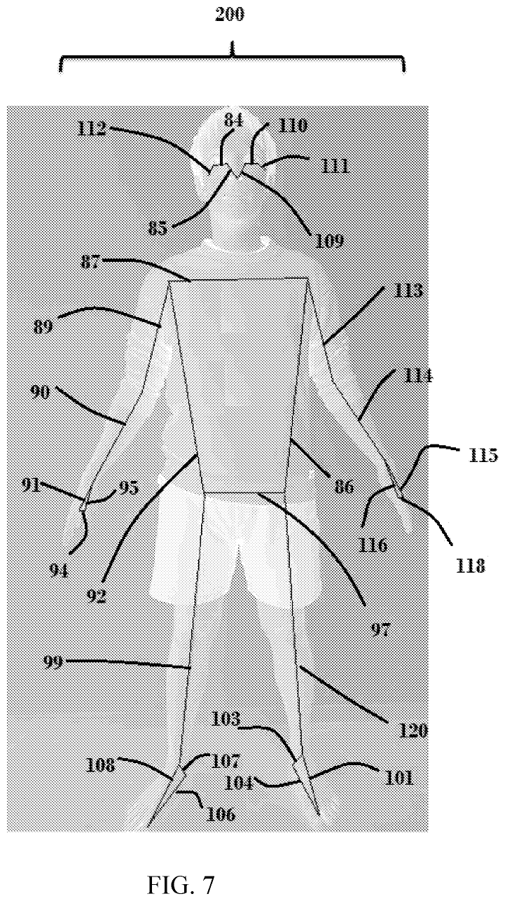

Referring FIG. 5 subject 200's pose landmark points 70 are illustratively shown for a subject, such as subject 200's nose 48, left eye inner 49, left eye 50, left eye outer 51, right eye inner 52, right eye 53, right eye outer 54, left ear 55, right ear 56, left mouth 57, mouth right 58, left shoulder 59, right shoulder 60, left elbow 61, right elbow 62, left wrist 63, right wrist 64, left pinky 65, right pinky 66, left index length of user 200's finger 67 and right index finger 68 and left thumb 69 and right thumb 71 and left hip 72 and right hip 73 and left knee 75 and right knee 76 and left ankle 77 and right ankle 78 and left heel 79 and right heel 80 and left foot index 81 and right foot index 82. At least one pose landmark point, as illustrated under pose landmarks 70, may be used as part of the development of measurements 13 for subject 200's body part measurements of FIG. 3, such as equivalent body part circumferences. In the current exemplary embodiment, the locating of at least one pose point of pose landmarks 70 of FIG. 5 may be performed by locating subject 200's pose landmark points via a computer routine and/or program, such as MediaPipe, and others not mentioned here. At least a portion of pose points 70 is then applied to at least one of subject 200's image 1, 2, 43, 44, 46, 47 of FIGS. 1-4 to develop the subject 200's line segmented side image of FIG. 6 and the line segmented frontal image of FIG. 7 where line segments are drawn between pose landmark points 70 as shown. Pose landmarks or pose landmark points may represent any feature of subject 200's body, such as to identify a given body part, joint, vertex and/or body part feature.

Referring FIG. 6, line segments 84, 85, 86, 87, 89, 90, 91, 92, 93, 94, 95, 97, 99, 101, 103, 104, 106, 107, 108, others not mentioned, here are shown as applied to subject 200's side image 1 of FIG. 1, connecting pose landmark points, such as represented by pose landmark points 70 of FIG. 5.

Referring FIG. 7, line segments such as line segments 84, 85, 86, 87, 89, 90, 91, 92, 94, 95, 97, 99, 101, 103, 104, 106, 107, 108, 112, 113, 114, 115, 116, 118, 120, others not mentioned here, are as applied to frontal image 2 of FIGS. 2, others not mentioned, connecting pose landmark points, such as representative pose landmark points 70 of FIG. 5. At least one line segment as illustrated in FIGS. 6 and 7 may be used as part of the development of measurements 13 for subject 200 of FIG. 3, such as equivalent circumference body part measurements.

For each of the pose landmarks, such as pose landmarks 70, a line segment originates at a given pose landmark or point, and is drawn to at least one of a body part contour edge and pose landmark point to get linear distance measurements for subject 200, as shown in FIGS. 5, 6 and 7. These linear distance measurements for subject 200's body are developed by scaling linear measurements using said subject's height input, such as subject 200's height as determined by the image height of subject 200 detected from the body height segment determined from images of FIGS. 1, 2, 4A-4D, 6 and 7. Additionally, the subject's height may be entered as part of subject 200's entries as part of at least one of biographic data 21 and user's questionnaire 20 of FIGS. 3 and 13 through 17. The prediction of the at least one of a measurement and body part circumference measurement of the subject 200's body may be based on at least one of said linear measurements and subject's weight and gender is then performed by system and method 100 of FIG. 3, such as through a computer routine such as a the commercially available XGBoost software, others not mentioned here.

In this first exemplary embodiment, histories of numerous subject's real life physical linear measurements and were used in conjunction with the line segment lineal measurement predictions determined by system and method 100 for improved accuracy. In addition, artificial intelligence methods such as the creation of an Application Programing Interface and/or API were used to control features used for training system and method 100. Additionally, the creation of control features used for training system and method 100 were developed in the user interface, such as UI 22 of FIG. 3. The output body part measurements, such as at least one of equivalent circumference measurements of subject 200's neck and chest waist and hip and thigh and calf and elbow and wrist and knee and ankle, determined through only two image inputs, such as image 1 and 2 of FIGS. 1 and 2, along with the user's biographic data of gender, height and weight are therefore the only input requirements for system and method 100 to function and provide at least one of an exercise routine and a nutrition routine. A user of system and method 100 may also be able to enter actual measurement of subject 200's body lineal and circumference measurements into system and method 100, such as via HMI 22, to compare and adjust the predicted lineal and circumference equivalence body part measurements and/or method determined by system and method 100.

Referring FIG. 8, boundaries are applied to subject 200's side image, such as user 200's side image 43 of FIG. 4A. FIG. 8 further illustrates upper boundary 121 and lower boundary 137 and left boundary 123 and right boundary 122, as well as chest 125 and wrist 134 and waist 127 and hips 128 and thigh 130 and calf 132 and ankle 135, others not mentioned here as applied to image 43.

Referring FIG. 9, boundaries are illustrated as applied to subject 200's image, such as an image provided by frontal image 46 of FIG. 4C. FIG. 9 shows upper boundary 139 and lower boundary 154 and left boundary 142 and right boundary 140, as well as chest 143 and wrist 145 and waist 146 and hips 148 and thigh 147 and the 150 and calf 151 and ankle 152 as applied to frontal image 46 of FIG. 46. At least one boundary as illustrated in FIGS. 8 and 9 may be used as part of the development of measurements 13 for subject 200's body of FIG. 3. The measurements as shown in FIGS. 8 and 9 are scaled from the subject's height and used by system and method 100 to predict subject 200's body part circumference and/or equivalent circumference measurements.

In this first exemplary embodiment, FIG. 10 is used to further illustrative ten locations for user 200's body part circumference measurements, such as scaled to user 200 in FIGS. 8 and 9, to develop user 200's predicted measurements 13 of FIG. 3, such as applied to data developed from subject 200's frontal image 2 of FIG. 2, comprising at least one of neck measurement 155 and chest measurement 158 and elbow measurement 157 and wrist measurement 162 and subject 200's waist measurement 160 and hips measurement 161 and thigh measurement 163 and knee measurement 171 and calf measurement 172 and ankle measurement 173 and others not mentioned here.

In this first exemplary embodiment, FIG. 11 illustratively shows ten locations for user 200's body part measurements, such as scaled and illustrated in FIGS. 8 and 9, to develop user 200's predicted measurements 13 of FIG. 3, such as applied to data developed from subject 200's side image 1 of FIG. 1, comprising at least one of neck measurement 175 and chest measurement 176 and elbow measurement 177 and wrist measurement 181 and subject 200's waist measurement 180 and hips measurement 179 and thigh measurement 183 and knee measurement 185 and calf measurement 186 and ankle measurement 188 and others not mentioned here. At least one measurement and measurement location as illustrated in FIGS. 8, 9, 10 and 11 may be used as part of the development of circumference measurements 13 for subject 200's body of FIG. 3.

Referring FIG. 12, predicted circumference measurements 13, such as used by system and method 100 of FIG. 3 for subject 200's body geometry are provided for at least one of subject 200's predicted neck circumference 189 and predicted chest circumference 190 and predicted waist circumference 191 and predicted hip circumference 192 and predicted thigh circumference 193 and calf predicted circumference 194 and predicted elbow circumference 195 and predicted wrist circumference 197 and predicted knee circumference 199 and predicted ankle circumference 201. At least one measurement illustrated in FIG. 12 may be used in determination of at least one of subject 200's fitness score 45 and wellness score 480 and workout score determination 475, others not mentioned here of FIG. 3.

In the current exemplary embodiment, referring FIGS. 3, 13, 14, 15, 16, 17, at least one of biometric data 21 and user questionnaire 20 is developed by at least one answer of user 200's answer of health history 204 and exercise and goals 205 and nutrition 206 and lifestyle 207 comprising at least one of high blood pressure yes answer 214 and high blood pressure no answer 216 and high cholesterol type yes answer 217 and high cholesterol type no answer 219 and diabetes yes answer 220 and diabetes no answer 221 and acid reflux yes answer 222 and acid reflux no answer 223 and heart disease yes answer 225 and heart disease no answer 226 and asthma yes answer 227 and asthma no answer 228 and medication yes answer 229 and medication no answer 231 and shoulder pain answer 233 and back pain answer 234 and knee pain answer 236 and other pain answer 237 and exercise location home answer 238 and exercise location gym answer 240 and exercise goal lose weight answer 241 and exercise goal build muscle answer 242 and weight training level highly advanced answer 243 and weight training level advanced answer 245 and weight training level average answer 246 and weight training level beginner answer 247 and cardio fitness level great answer 248 and cardio fitness level above average 250 and cardio fitness level average answer 251 and cardio fitness level poor answer 252 and exercise frequency level 5 days or more a week answer 253 and exercise frequency level 4 days a week answer 254 and exercise frequency level 3 days a week answer 255 and exercise frequency level twice a week 256 and exercise frequency once a week answer 257 and exercise frequency level I don't exercise at all answer 258 and workout intensity level answer 264 and workout intensity level very high answer 259 and workout intensity level high answer 261 and workout intensity level medium answer 262 and workout intensity level answer 263 and workout intensity level very low answer 265 and workout difficulty level answer 266 and workout difficulty level very easy answer 267 and workout difficulty level easy answer 268 and workout difficulty level just right answer 269 and workout difficulty level hard answer 270 and workout difficulty level very hard answer 272 and nutrition section 274 answers and nutrition very good answer 275 and nutrition good answer 276 and nutrition average human answer 277 and nutrition poor answer 278 and nutrition very poor answer 279 and meal frequency level answer 280 and meal frequency level 5 or more times a day answer 282 and meal frequency level 4 times a day answer 284 and meal frequency level 3 times a day answer 285 and meal frequency level twice a day answer 286 and meal frequency level once a day answer 287 and meal preference level vegetarian answer 291 and meal preference level gluten-free answer 292 and meal preference level no preference answer 293 and social support level very good answer 298 and social support level good answer 290 and social support level average answer 299 and social support level poor answer 300 and social support level very poor answer 302 and financial level extremely happy answer 306 and financial level very happy answer 307 and financial level happy answer 308 and financial level unhappy answer 309 and financial level very unhappy answer 310 and stress level no stress answer 311 and stress level average stress answer 312 and stress level very stressed answer 313 and sleep level above normal answer 317 and sleep level answer normal answer 318 and sleep level below normal answer 319 and confidence level extremely confident answer 325 and confidence level very confident answer 326 and confidence level confident answer 327 and confidence level unconfident answer 328 and confidence level very unconfident answer 329 and relationship level extremely happy answer 331 and relationship level very happy answer 332 and relationship level happy answer 333 and relationship level unhappy answer 334 and relationship level very unhappy answer 335 and alcohol I don't drink alcohol answer 337 and alcohol 124 drinks a week answer 338 and alcohol 5 to 10 drinks a week 339 and alcohol 11 to 20 drinks a week 340 and alcohol 20 or more drinks a week answer 341 and smoking yes answer 344 and smoking no answer 345 and others not mentioned here. In this first exemplary embodiment, a location relevant to subject 200 may comprise at least one of said subject 200's whereabouts and a home location and a gymnasium location and an exercise location and any other location information relevant to subject 200's ability to perform exercise.

In this first exemplary embodiment, referring FIGS. 3 and 19, body fat percentage 19 is used in system and method 100 of FIG. 3, such as by characterizer 15 of FIG. 3, whereby said body fat percentage process 19 utilizes at least one portion of biometric data 21 and image data derived from subject's side image 1 and subjects frontal image 2 of FIGS. 1 and 2 respectively. In this first exemplary embodiment, body fat percentage process 19 is developed by processing of at least one of body fat percentage calculator 368 and body volume less organs calculator 370 and body key points determinator 376 and body part volume calculator 372 and human organ volume adjuster 374 and volume to body fat percentage converter 378 and body fat accuracy tester 380 and inaccuracy processor 382 and body key points adder 384 and muscle factor process 386 and fluid factor process 388 and accuracy tester 390, others not mentioned here, as illustrated in FIG. 19 and as described herein. Determining, as in this first exemplary embodiment, may be comprised of calculating, estimating, predicting, extrapolating, others not mentioned here.

Referring FIGS. 20, 21, 22, 23, 24 and 25, at least one portion of the measurements process 400 may be utilized by at least one of system and method 100 and characterizer 15 and process 33 and fitness score 45 and wellness score 480 and workout score 475 of FIG. 3 and others not mentioned here. Referring FIGS. 20, 21, 22, 23, 24 and 25, measurements process 400 may utilize at least one of body part measurements analysis 402 comprising measurement factoring 403 and calculations 19, 406, 407, 408 409, 410, 411, 412, 413, 414, 415, 416, 417, 418, 420, 422, 423, 424, 426, 428, 430, 432, 433, 435, 437, 438, and 440, others not mentioned here to develop and/or characterize any aspect of user 200's physical fitness level.

Referring FIGS. 26, 27, 28, 29, fitness score 45 such as used by at least a portion of system and method 100 and wellness score 480 and workout score 475 and nutrition determinator 38 of FIG. 3 may comprise at least one portion of fitness score 441 and fitness score calculation 442 and male waist circumference level 444 and female waist circumference 446 and body mass index level 447 and body fat percentage level 448 and female body fat percentage level 449 and 451 and male LBMI to FBMI ratio 453 and female LBMI to FBMI ratio 455 male waist to height ratio level 456 and female waist to height ratio 458 and cardio fitness level 460 and weight training level 462 and rationale number 464 and age level 465 and workout intensity level 466 and workout difficulty level 477, others not mentioned here. LBMI as used in this first exemplary embodiment relates to lean body mass index calculation, and FBMI relates to fat body mass index. In the current exemplary embodiment, a waist to height ratio, such as waist to height ratio level 456 and 458, refer to at least one of subject 200's waist measurement and waist circumference divided by subject 200's height. Alternately, a waist to height ratio, such as waist to height ratio level 456 and 458, may refer to at least one of subject 200's height measurement divided by at least one of subject 200's waist measurement and waist circumference. Waist circumference refers to at least one of subject 200's waist circumference and waist equivalent circumference. In this current exemplary embodiment, weight training level may refer to any quantitative and/or qualitative determination regarding at least one of subject 200's strength level and weight lifting experience and weightlifting goals and strength training and others not mentioned here. As defined herein, a body measurement of a body part of subject 200, may refer to a circumference and an equivalent circumference.

Referring FIGS. 30 and 31, workout score 475 such as used by at least one portion of system and method 100 and process 33 and exercise database 34 and others not mentioned here of FIG. 3, comprises at least one of workout score rationale and calculation 469 and age level 470 and workout intensity level 471 and workout difficulty level 473 and exercise frequency level 476 and others not mentioned here as shown and referenced herein.

Referring FIGS. 32, 33, 34, develop wellness score 480 such as used by at least one portion of system and method 100 and process 33 and nutrition determinator 38 and others not mentioned here of FIG. 3. Wellness score may develop based on at least one of wellness score calculation 514 and fitness score level analysis 515 and nutrition level analysis 516 and social support level analysis 517 and financial level analysis 518 and stress level analysis 519 and sleep level analysis 520 and confidence level analysis 521 and relationship level analysis 522 and health factor levels analysis 523 and others not mentioned here as shown and referenced herein.

Referring FIG. 35, exercise database 34 such as used by system and method 100 of FIG. 3 may comprise filterable data such as at least one of identifier 481 and program name 482 and program location 483 and program equipment 486 and program workout score level 484 and program fitness goal 485 and workout schedule 487 and injury input 488 and others not mentioned here as shown and referenced herein. Additionally, referring FIG. 36, exercise database 34 may also comprise filterable data such as at least one of workout ID 492 and exercise name 494 and number of sets 495 and number of reps per set 496 and time/duration 497 and rest between sets 498 and cardio time duration 499 and level of exertion 501 and workout score level 503.

Referring FIG. 37, Nutrition Determination Formulas 38 comprises a male and female calorie suggestion formula considering a male and female weight factor and a male and female high factor as well as a weight loss goal and build muscle goal to determine the calorie recommendation for a given male and female such as nutrition determination 40 by system and method 100 of FIG. 3. Nutrition formula determination 38 of FIG. 37 also provides a recommended protein calculation for both a male and a female based on the subject's weight. Further determination formula 38 provides a recommended a ounces of daily water intake based on the user's weight.

What is claimed is:

1. A system to determine the physical fitness level of a subject and prescribe an exercise routine for said subject, the system comprising:
a non-transitory computer-readable medium encoded with computer executable instructions that, as a result of being executed by a processor coupled to a display and an image capture system, whereby said image capture system captures a subject frontal image and a subject side image, and said processer executes a method for determining a physical fitness level for said subject and assigns an exercise routine based on said physical fitness level for said subject, method comprising:
using a human machine interface, whereby a subject's biographic data is entered therethrough said human machine interface and said biographic data is at least one of stored thereupon and retrieved therefrom said non-transitory computer-readable medium;
using a body part measurement determined from at least one portion of said subject's torso and neck and limbs from said subject's side image and said subject's frontal image;
characterizing a physical fitness level for said subject's body based on said subject's biographic data and at least one body part measurement of said subject's body developed therefrom at least one of said subject's side image and said subject's frontal image;
using an exercise routine records database comprising a plurality of exercise routine records, each said exercise routine record being identifiable, whereby said processor performs executable instructions to filter said exercise routine records of said exercise routine records database based on filter criteria comprising at least one of the subject's said biographic data and subject's said determined physical fitness level to output at least one exercise routine record for said subject; and
communicating said at least one exercise routine record via said display.

2. The system of claim 1, whereby at least one portion of said subject's physical fitness level comprises a body fat percentage calculation for said subject based on the subject's said at least one body part measurement.

3. The system of claim 1, whereby the said body part measurement of said subject's side image and said subject's frontal image is at least one of a circumference measurement and an equivalent circumference measurement of a body part of said subject.

4. The system of claim 2, whereby the said body part measurement of the said subject's side image and said subject's frontal image is at least one of a circumference measurement and an equivalent circumference measurement of a body part of said subject.

5. The system of claim 1, whereby at least a portion of said subject's biographic data comprises said subject's height and weight and gender.

6. The system of claim 2, whereby said subject's biographic data comprises said subject's height and weight and gender.

7. The system of claim 3, whereby said subject's biographic data comprises said subject's height and weight and gender.

8. The system of claim 4, whereby said entered subject's biographic data comprises said subject's height and weight and gender.

9. The system of claim 7, whereby said physical fitness level is based thereupon said at least one of said circumference measurement and an equivalent circumference measurement comprising at least one of said subject's waist and chest and thigh and hip.

10. The system of claim 8, whereby said physical fitness level is based thereupon said at least one of said circumference measurement and an equivalent circumference measurement comprising at least one of said subject's waist and chest and thigh and hip.

11. The system of claim 9, whereby said physical fitness level is based thereupon at least one of said circumference measurement and an equivalent circumference measurement further comprising at least one of said subject's bicep and elbow and wrist and calf and ankle and knee.

12. The system of claim 10, whereby said body fat percentage calculation is based thereupon at least one of said body part measurement and circumference further comprising at least one of said subject's bicep and elbow and wrist and calf and ankle and knee.

13. The system of claim 9, whereby said at least one exercise routine record for said subject is identified by filtering said exercise routine database by filter criteria comprising at least one of an exercise routine record identifier and said subject's equipment and schedule and location and age and injury status.

14. The system of claim 11, whereby said at least one exercise routine record for said subject is identified by filtering said exercise routine database using filter criteria comprising at least one of an exercise routine record identifier and said subject's equipment and schedule and location and age and injury status.

15. The system of claim 12, whereby said at least one exercise routine record for said subject is identified by filtering said exercise routine database using filter criteria comprising at least one of an exercise routine record identifier and said subject's equipment and schedule and location and age and injury status.

16. The system of claim 1, further determining at least one nutrition routine for said subject comprising at least one of calorie intake and protein intake and daily water intake for the subject, whereby said computer performs executable instructions to determine said nutrition routine based on at least one of said subject's biographic data and subject's said physical fitness level and communicating said at least one nutrition routine via said display.

17. The system of claim 14, further determining at least one nutrition routine for said subject comprising at least one of calorie intake and protein intake and daily water intake for the subject, whereby said computer performs executable instructions to determine said nutrition routine based on at least one of said subject's biographic data and subject's said physical fitness level and communicating said at least one nutrition routine via said display.

18. The system of claim 15, further determining at least one nutrition routine for said subject comprising at least one of calorie intake and protein intake and daily water intake for the subject, whereby said computer performs executable instructions to determine said nutrition routine based on at least one of said subject's biographic data and subject's said physical fitness level and communicating said at least one nutrition routine via said display.

19. The system of claim 17, whereby filter criteria used to identify said at least one of said subject's at least one exercise routine further comprises at least one of said subject's waist measurement and said subject's waist circumference and subject's waist to height ratio and subject's cardio fitness level and subject's weight training level.

20. The system of claim 18 whereby filter criteria used to identify said at least one of said subject's at least one exercise routine further comprises at least one of said subject's waist measurement and said subject's waist circumference and subject's waist to height ratio and subject's cardio fitness level and subject's weight training level.

21. A system to determine the physical fitness level of a subject and prescribe an exercise routine for said subject, the system comprising:
a non-transitory computer-readable medium encoded with computer executable instructions that, as a result of being executed by a processor coupled to a display and an image capture system, whereby said image capture system captures a subject's frontal image and a subject's side image, and said processer executes a method for determining a physical fitness level for said subject and identifying an exercise routine based on said physical fitness score for said subject, method comprising:
using a human machine interface, whereby said subject's height and weight and gender are entered therethrough said human machine interface and are at least one of processed by said processor and stored thereupon said non-transitory computer-readable medium encoded;
predicting body part equivalent circumference measurements for said subject's waist and chest and thigh and hips from said subject's side image and said subject's frontal image data;
characterizing said subject's physical fitness score based on said subject's body part equivalent circumference measurements of said subject's waist and chest and thigh and hip equivalent circumference measurements and said subject's height and weight and gender;
using an exercise routine records database comprising a plurality of exercise routine records, each of said exercise routine record being identifiable, whereby said processor performs executable instructions to filter said exercise routine records of said exercise routine records database based on filter criteria of said subject's physical fitness score to identify at least one exercise routine record for said subject; and
communicating said at least one exercise routine record via said display.

22. The system of claim 21, further comprising:
predicting body part equivalent circumference measurements for at least one of said subject's neck and calf and bicep and elbow and wrist and ankle from subject's side image and said subject's frontal image data and;
determining a body fat percentage calculation for said subject's body and further basing said subject's physical fitness score on at least one of said body fat percentage calculation and said subject's body part equivalent circumference measurements.

23. The system of claim 22, further determining at least one nutrition routine for said subject comprising at least one of calorie intake and protein intake and daily water intake for the subject, whereby said computer performs executable instructions to determine said nutrition routine based on at least one of said subject's biographic data and subject's said physical fitness level and communicating said at least one nutrition routine via said display.

24. The system of claim 22, whereby said at least one of said subject's body part equivalent circumference measurements is determined through image processing at least one portion of the said subject's side image and said subject's frontal image, said image processing comprising:
producing a subject's extracted side image from instance segmenting said subject's side image by extracting only said subject's body from said subjects side image;
producing a subject's extracted frontal image from instance segmenting said subject's frontal image by extracting only said subject's body from said subject's frontal image;
producing a subject's side posture image with inner and outer edges from said subject's extracted side image;
producing a subject's frontal posture image with inner and outer edges from said subject's extracted frontal image; and
locating at least a portion of the outer contours of said subject's extracted images through edge detection.

25. The system of claim 23, whereby said at least one of said subject's body part equivalent circumference measurements is determined through image processing at least one portion of the said subject's side image and said subject's frontal image, said image processing comprising:
producing a subject's extracted side image from instance segmenting said subject's side image by extracting only said subject's body from said subjects side image;
producing a subject's extracted frontal image from instance segmenting said subject's frontal image by extracting only said subject's body from said subject's frontal image;
producing a subject's side posture image with inner and outer edges from said subject's extracted side image;
producing a subject's frontal posture image with inner and outer edges from said subject's extracted frontal image; and
locating at least a portion of the outer contours of said subject's extracted images through edge detection.

26. The system of claim 24, whereby said image processing of at least one portion of the said subject's side image and said subject's frontal image further comprises:
locating pose landmarks for said subject's side image and frontal image;
drawing at least one line segment that intersects at least two of said pose landmarks to develop at least one linear measurement of said at least one line segment;
scaling at least one of said linear measurements using said subject's height;
predicting the said least one body part circumference measurement of the subject's body based on said at least one linear measurement.

27. The system of claim 25, whereby said image processing of at least one portion of the said subject's side image and said subject's frontal image further comprises:
locating pose landmarks for said subject's side image and frontal image;
drawing at least one line segment that intersects at least two of said pose landmarks to develop at least one linear measurement of said at least one line segment;
scaling at least one of said linear measurements using said subject's height;
predicting the said least one body part circumference measurement of the subject's body based on said at least one linear measurement.
